(19) United States
(12) Patent Application Publication
(10) Pub. No.: US 2006/0116389 A1
(43) Pub. Date: Jun. 1, 2006

(54) Antibacterial oxazolidinones

(75) Inventors: Michael Barry Gravestock, Waltham, MA (US); Neil James Hales, Macclesfield (GB); Paul Turner, Macclesfield (GB)

Correspondence Address: ASTRAZENECA R&D BOSTON, 35 GATEHOUSE DRIVE, WALTHAM, MA, 02451-1215, US

(73) Assignee: ASTRA ZENECA AB, SODERTAJE (SE)

(21) Appl. No.: 10/539,484

(22) PCT Filed: Dec. 15, 2003
(86) PCT No.: PCT/GB03/05444

§ 371 Date: Jun. 17, 2005

(30) Foreign Application Priority Data
Dec. 19, 2002 (GB) 0229522.8

(51) Int. Cl. A61K 31/513; A61K 31/506; A61K 31/4439; A61K 31/655; C07D 413/02

(52) U.S. Cl. 514/269; 544/314; 544/331; 546/271.4; 514/275; 514/340; 514/151

(57) ABSTRACT

A compound of the formula (I), or a pharmaceutically-acceptable salt, or in-vivo hydrolysable ester thereof: formula (I) wherein C is selected from D and E, formula (II) R₂a, R₆a, and R₃a are independently selected from for example H, CF₃, Me and Et; R₂b and R₆b are independently selected from for example H, F, CF₃, Me and Et; R₁b is —NRz-Z wherein Rz is for example hydrogen and Z is a 5- or 6-membered heteroaryl ring; R₄ is for example an optionally substituted 5- or 6-membered heterocyclic ring system. Methods for making compounds of the formula (I), compositions containing them and their use as antibacterial agents are also described.

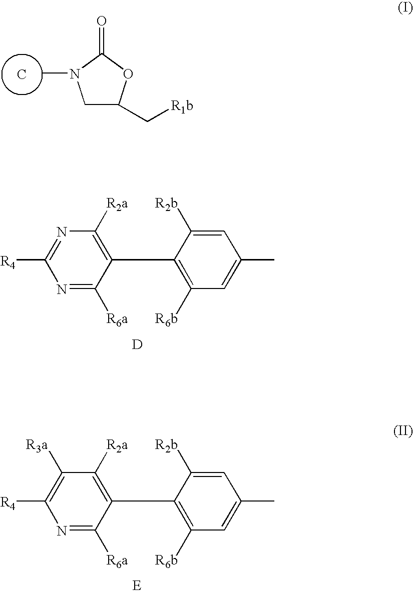

The present invention relates to antibiotic compounds and in particular to antibiotic compounds containing substituted oxazolidinone rings. This invention further relates to processes for their preparation, to intermediates useful in their preparation, to their use as therapeutic agents and to pharmaceutical compositions containing them.

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus (MRSA), methicillin resistant coagulase negative staphylococci (MRCNS), penicillin resistant Streptococcus pneumoniae and multiply resistant Enterococcus faecium.

The major clinically effective antibiotic for treatment of such resistant Gram-positive pathogens is vancomycin. Vancomycin is a glycopeptide and is associated with various toxicities including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H.influenzae and M.catarrhalis.

Certain antibacterial compounds containing an oxazolidinone ring have been described in the art (for example, Walter A. Gregory et al in J. Med. Chem. 1990, 33, 2569-2578 and 1989, 32(8), 1673-81; Chung-Ho Park et al in J. Med. Chem. 1992, 35, 1156-1165). Bacterial resistance to known antibacterial agents may develop, for example, by (i) the evolution of active binding sites in the bacteria rendering a previously active pharmacophore less effective or redundant, and/or (ii) the evolution of means to chemically deactivate a given pharmacophore, and/or (iii) the evolution of efflux pathways. Therefore, there remains an ongoing need to find new antibacterial agents with a favourable pharmacological profile, in particular for compounds containing new, more potent, pharmacophores.

Accordingly the present invention provides a compound of the formula (I), or a pharmaceutically-acceptable salt, or an in-vivo-hydrolysable ester thereof, 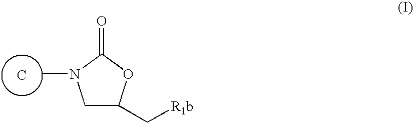
wherein C is selected from D and E, 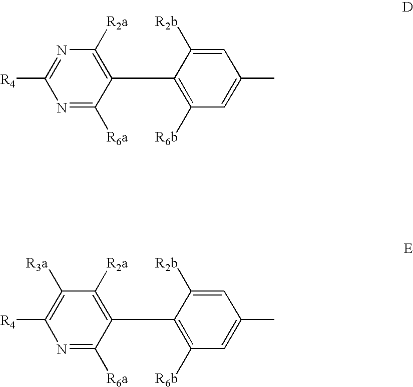
wherein in D and E the phenyl ring is attached to the oxazolidinone in (I);

R₁b is —NRz-Z wherein Rz is hydrogen, (1-6C)alkyl or —COOR₅ wherein R₅ is (1-6C) alkyl optionally substituted by one or more chlorine atoms;

Z is HET-1 wherein

HET-1 is selected from HET-1A and HET-1B wherein:

HET-1A is a C-linked 5-membered heteroaryl ring containing 2 to 4 heteroatoms independently selected from N, O and S; which ring is optionally substituted on a C atom by an oxo or thioxo group; and/or which ring is optionally substituted on any available C atom by one or two substituents selected from RT as hereinafter defined and/or on an available nitrogen atom, (provided that the ring is not thereby quaternised) by (1-4C)alkyl;

HET-1B is a C-linked 6-membered heteroaryl ring containing 2 or 3 nitrogen heteroatoms, which ring is optionally substituted on a C atom by an oxo or thioxo group; and/or which ring is optionally substituted on any available C atom by one, two or three substituents selected from RT as hereinafter defined and/or on an available nitrogen atom, (provided that the ring is not thereby quaternised) by (1-4C)alkyl;

RT is selected from a substituent from the group:

(RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano and nitro; or

(RTa2) (1-4C)alkylamino, di-(1-4C)alkylamino, and (2-4C)alkenylamino; or RT is selected from the group

(RTb1) (1-4C)alkyl group which is optionally substituted by one substituent selected from hydroxy, (1-4C)alkoxy, (1-4C)alkylthio, cyano and azido; or

(RTb2) (1-4C)alkyl group which is optionally substituted by one substituent selected from (2-4C)alkenyloxy, (3-6C)cycloalkyl, and (3-6C)cycloalkenyl; or RT is selected from the group

(RTc) a fully saturated 4-membered monocyclic ring containing 1 or 2 heteroatoms independently selected from O, N and S (optionally oxidised), and linked via a ring nitrogen or carbon atom;

and wherein at each occurrence of an RT substituent containing an alkyl, alkenyl, alkynyl, cycloalkyl or cycloalkenyl moiety in (RTa1) or (RTa2), (RTb1) or (RTb2), or (RTc) each such moiety is optionally substituted on an available carbon atom with one, two, three or more substituents independently selected from F, Cl, Br, OH and CN;

R₂a and R₆a are independently selected from H, CF₃, OMe, SMe, Me and Et;

R₂b and R₆b are independently selected from H, F, Cl, CF₃, OMe, SMe, Me and Et;

R₃a is selected from H, (1-4C)alkyl, cyano, Br, F, Cl, OH, (1-4C)alkoxy, —S(O)ₙ(1-4C)alkyl (wherein n=0, 1, or 2), amino, (1-4C)alkylcarbonylamino, nitro, —CHO, —CO(1-4C)alkyl, —CONH₂ and —CONH(1-4C)alkyl;

R₄ is selected from R₄a and R₄b wherein

R₄a is selected from azido, —NR₇R₈, OR₁₀, (1-4C)alkyl, (1-4C)alkoxy, (3-6C)cycloalkyl, —(CH₂)ₖ—R₉, AR1, AR2, (1-4C)alkanoyl, —CS(1-4C)alkyl, —C(═W)NRvRw [wherein W is O or S, Rv and Rw are independently H, or (1-4C)alkyl], —(C═O)₁—R₆, —COO(1-4C)alkyl, —C═OAR1, —C═OAR2, —COOAR1, —S(O)n(1-4C)alkyl (wherein n=1 or 2), —S(O)pAR1, —S(O)pAR2 and C(═S)O(1-4C)alkyl; wherein any (1-4C)alkyl chain may be optionally substituted by (1-4C)alkyl, cyano, hydroxy or halo;

p=0, 1 or 2;

R₄b is selected from HET-3;

R₆ is selected from hydrogen, (1-4C)alkoxy, amino, (1-4C)alkylamino and

hydroxy(1-4C)alkylamino;

k is 1 or 2;

l is 1 or 2;

R₇ and R₈ are independently selected from H and (1-4C)alkyl, or wherein R₇ and R₈ taken together with the nitrogen to which they are attached can form a 5-7 membered ring optionally with an additional heteroatom selected from N, O, S(O)n (wherein n=1 or 2) in place of 1 carbon atom of the so formed ring; wherein the ring may be optionally substituted by one or two groups independently selected from (1-4C)alkyl, (3-6C)cycloalkyl, (1-4C)alkanoyl, —COO(1-4C)alkyl, —S(O)n(1-4C)alkyl (wherein n=1 or 2), AR1, AR2, —C═OAR1, —C═OAR2, —COOAR1, —CS(1-4C)alkyl, —C(═S)O(1-4C)alkyl, —C(═W)NRvRw [wherein W is O or S, Rv and Rw are independently H, or (1-4C)alkyl], —S(O)pAR1 and —S(O)pAR2; wherein any (1-4C)alkyl, (3-6C)cycloalkyl or (1-4C)alkanoyl group may be optionally substituted (except on a carbon atom adjacent to a heteroatom) by one or two substituents selected from (1-4C)alkyl, cyano, hydroxy, halo, amino, (1-4C)alkylamino and di(1-4C)alkylamino; p=0, 1 or 2;

R₉ is independently selected from R₉a to R₉d below:

R₉a: AR1, AR2, AR2a, AR2b, AR3, AR3a, AR3b, AR4, AR4a, CY1, CY2;

R₉b: cyano, carboxy, (1-4C)alkoxycarbonyl, —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1, —CS(1-4C)alkyl and —C(═S)O(1-4C)alkyl; wherein any alkyl, alkanoyl or cycloalkyl may itself optionally be substituted by cyano, hydroxy or halo)], ethenyl, 2-(1-4C)alkylethenyl, 2-cyanoethenyl, 2-cyano-2-((1-4C)alkyl)ethenyl, 2-nitroethenyl, 2-nitro-2-((1-4C)alkyl)ethenyl, 2-((1-4C)alkylaminocarbonyl)ethenyl, 2-((1-4C)alkoxycarbonyl)ethenyl, 2-(AR1)ethenyl, 2-(AR2)ethenyl, 2-(AR2a)ethenyl;

R₉c: (1-6C)alkyl

{optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, (1-4C)alkylcarbonyl, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], and amino; and/or optionally substituted by one group selected from carboxy, phosphonate [phosphono, —P(O)(OH)₂, and mono- and di-(1-4C)alkoxy derivatives thereof], phosphinate [—P(OH)₂ and mono- and di-(1-4C)alkoxy derivatives thereof], cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, (1-4C)alkylamino, di((1-4C)alkyl)amino, (1-6C)alkanoylamino-, (1-4C)alkoxycarbonylamino-, N-(1-4C)alkyl-N-(1-6C)alkanoylamino-, —C(═W)NRvRw [wherein W is O or S, Rv and Rw are as hereinbefore defined], (═NORv) wherein Rv is as hereinbefore defined, (1-4C)alkylS(O)ₚNH, (1-4C)alkylS(O)ₚ-((1-4C)alkyl)N—, fluoro(1-4C)alkylS(O)ₚNH—, fluoro(1-4C)alkylS(O)ₚ((1-4C)alkyl)N—, (1-4C)alkylS(O)_{q}—, CY1, CY2, AR1, AR2, AR3, AR1-O—, AR2-O—, AR3-O—, AR1-S(O)_{q}—, AR2-S(O)_{q}—, AR3-S(O)_{q}—, AR1-NH—, AR2-NH—, AR3-NH— (p is 1 or 2 and q is 0, 1 or 2), and also AR2a, AR2b, AR3a and AR3b versions of AR2 and AR3 containing groups}; wherein any (1-4C)alkyl present in any substituent on R₉c may itself be substituted by one or two groups independently selected from cyano, hydroxy, halo, amino, (1-4C)alkylamino and di(1-4C)alkylamino, provided that such a substituent is not on a carbon adjacent to a heteroatom atom if present;

R₉d: R₁₄C(O)O(1-6C)alkyl- wherein R₁₄ is AR1, AR2, (1-4C)alkylamino, benzyloxy-(1-4C)alkyl or (1-10C)alkyl {optionally substituted as defined for (R9c)};

R₁₀ is selected from hydrogen, R₉c (as hereinbefore defined), (3-6C)alkanoyl and (1-4C)alkylsulfonyl;

HET-3 is selected from:

a) a 5-membered heterocyclic ring contining at least one nitrogen and/or oxygen in which any carbon atom is a C═O, C═N, or C═S group, wherein said ring is of the formula HET3-A to HET3-E below: 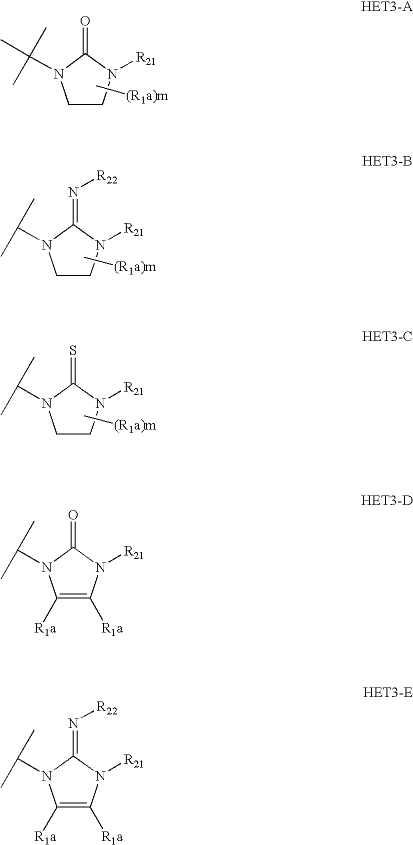
b) a carbon-linked 5- or 6-membered heteroaromatic ring containing 1, 2, 3, or 4 heteroatoms independently selected from N, O and S selected from HET3-F to HET3-Y below: 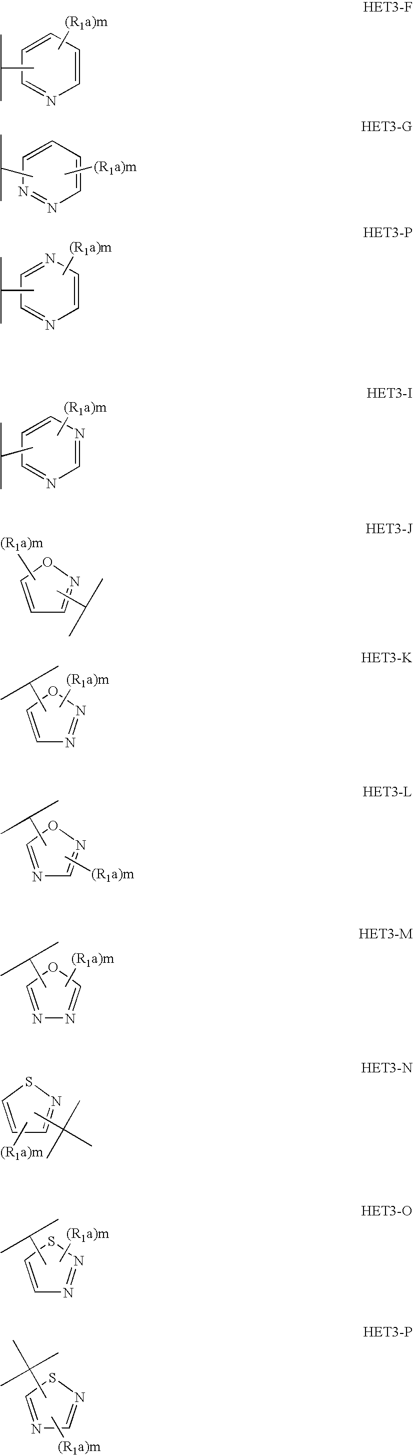 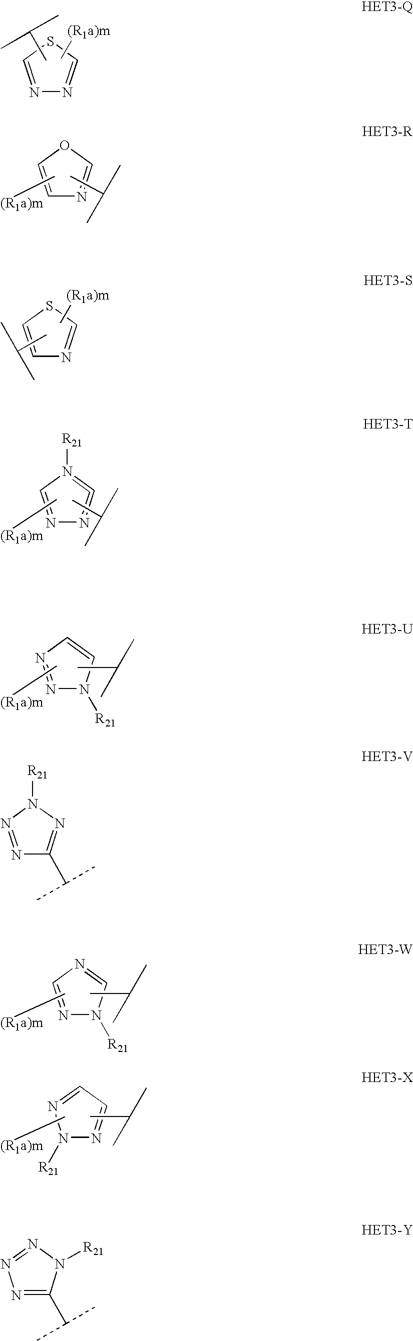
c) a nitrogen-linked 5- or 6-membered heteroaromatic ring containing 1, 2, 3, or 4 heteroatoms independently selected from N, O and S selected from HET3-Z to HET3-AH below: 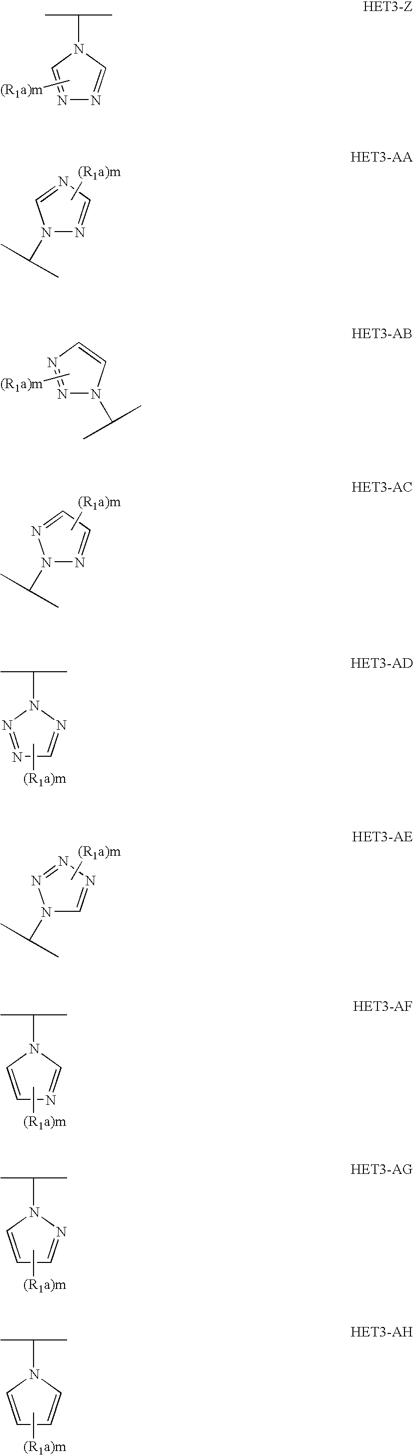
wherein in HET-3, R₁a is a substituent on carbon;
R₁a is independently selected from R₁a1 to R₁a5 below:
R₁a1: AR1, AR2, AR2a, AR2b, AR3, AR3a, AR3b, AR4, AR4a, CY1, CY2;
R₁a2: cyano, carboxy, (1-4C)alkoxycarbonyl, —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1, —CS(1-4C)alkyl) and —C(═S)O(1-4C)alkyl; wherein any (1-4C)alkyl, (1-4C)alkanoyl and (3-6C)cycloalkyl substituent may itself be substituted by cyano, hydroxy or halo, provided that, such a substituent is not on a carbon adjacent to a nitrogen atom of the piperazine ring], ethenyl, 2-(1-4C)alkylethenyl, 2-cyanoethenyl, 2-cyano-2-((1-4C)alkyl)ethenyl, 2-nitroethenyl, 2-nitro-2-((1-4C)alkyl)ethenyl, 2-((1-4C)alkylaminocarbonyl)ethenyl, 2-((1-4C)alkoxycarbonyl)ethenyl, 2-(AR1)ethenyl, 2-(AR2)ethenyl, 2-(AR2a)ethenyl;
R₁a3: (1-10C)alkyl
{optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, (1-4C)alkylcarbonyl, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], and amino; and/or optionally substituted by one group selected from carboxy, phosphonate [phosphono, —P(O)(OH)₂, and mono- and di-(1-4C)alkoxy derivatives thereof], phosphinate [—P(OH)₂ and mono- and di-(1-4C)alkoxy derivatives thereof], cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, (1-4C)alkylamino, di((1-4C)alkyl)amino, (1-6C)alkanoylamino-, (1-4C)alkoxycarbonylamino-, N-(1-4C)alkyl-N-(1-6C)alkanoylamino-, —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1, —CS(1-4C)alkyl and —C(═S)O(1-4C)alkyl], (═NORv) wherein Rv is as hereinbefore defined, (1-4C)alkylS(O)ₚNH—, (1-4C)alkylS(O)ₚ-((1-4C)alkyl)N—, fluoro(1-4C)alkylS(O)ₚNH—, fluoro(1-4C)alkylS(O)ₚ((1-4C)alkyl)N—, (1-4C)alkylS(O)_{q}—, CY1, CY2, AR1, AR2, AR3, AR1-O—, AR2-O—, AR3-O—, AR1-S(O)_{q}—, AR2-S(O)_{q}—, AR3-S(O)_{q}—, AR1-NH—, AR2-NH—, AR3-NH— (p is 1 or 2 and q is 0, 1 or 2), and also AR2a, AR2b, AR3a and AR3b versions of AR2 and AR3 containing groups); wherein any (1-4C)alkyl, (1-4C)alkanoyl and (3-6C)cycloalkyl present in any substituent on R₁a3 may itself be substituted by one or two groups independently selected from cyano, hydroxy, halo, amino, (1-4C)alkylamino and di(1-4C)alkylamino, provided that such a substituent is not on a carbon adjacent to a heteroatom atom if present;
R₁a4: R₁₄C(O)O(1-6C)alkyl- wherein R₁₄ is as hereinbefore defined for R₉d;
R₁a5: F, Cl, hydroxy, mercapto, (1-4C)alkylS(O)ₚ— (p=0, 1 or 2), —NR₇R₈ (wherein R₇ and R₈ are as hereinbefore defined) or —OR₁₀ (where R₁₀ is as hereinbefore defined);
m is 0, 1 or 2;
R₂₁ is selected from hydrogen, methyl [optionally substituted with cyano, trifluoromethyl, —C—WNRvRw (where W, Rv and Rw are as hereinbefore defined for R₁a3), (1-4C)alkoxycarbonyl, (1-4C)alkoxy-(1-4C)alkoxycarbonyl, (1-4C)alkoxy-(1-4C)alkoxy-(1-4C)alkoxycarbonyl, CY1, CY2, AR1, AR2, AR2a, AR2b (not linked through nitrogen) or AR3], (2-10C)alkyl [optionally substituted other than on a carbon attached to the HET-3 ring nitrogen with one or two groups independently selected from the optional subsituents defined for R₁a3] and R₁₄C(O)O(2-6C)alkyl-, wherein R₁₄ is as defined hereinbefore and wherein R₁₄C(O)O group is attached to a carbon other than the carbon attached to the HET-3 ring nitorogen;
R₂₂ is cyano, —COR₁₂, —COOR₁₂, —CONHR₁₂, —CON(R₁₂)(R₁₃), —SO₂R₁₂ (provided that R₁₂ is not hydrogen), —SO₂NHR₁₂, —SO₂N(R₁₂)(R₁₃) or NO₂, wherein R₁₂ and R₁₃ are as defined hereinbelow;
R₁₂ and R₁₃ are independently selected from hydrogen, phenyl (optionally substituted with one or more substituents selected from halogen, (1-4C)alkyl and (1-4C)alkyl substituted with one, two, three or more halogen atoms) and (1-4C)alkyl (optionally substituted with one, two, three or more halogen atoms), or for any N(R₁₂)(R₁₃) group, R₁₂ and R₁₃ may be taken together with the nitrogen to which they are attached to form a 5-7 membered ring optionally with an additional heteroatom selected from N, O, S(O)n in place of 1 carbon atom of the so formed ring; wherein the ring may be optionally substituted by one or two groups independently selected from (1-4C)alkyl (optionally substituted on a carbon not adjacent to the nitrogen by cyano, hydroxy or halo), (3-6C)cycloalkyl, (1-4C)alkanoyl, —COO(1-4C)alkyl, —S(O)n(1-4C)alkyl (wherein n=1 or 2), AR1, AR2, —C═OAR1, —C═OAR2, —COOAR1, —CS(1-4C)alkyl, —C(═S)O(1-4C)alkyl, —C(═W)NRvRw [wherein W is O or S, Rv and Rw are independently H, or (1-4C)alkyl], —S(O)pAR1 and —S(O)pAR2; wherein any (1-4C)alkyl chain may be optionally substituted by (1-4C)alkyl, cyano, hydroxy or halo; p=0, 1 or 2;
AR1 is an optionally substituted phenyl or optionally substituted naphthyl;
AR2 is an optionally substituted 5- or 6-membered, fully unsaturated (i.e with the maximum degree of unsaturation) monocyclic heteroaryl ring containing up to four heteroatoms independently selected from O, N and S (but not containing any O—O, O—S or S—S bonds), and linked via a ring carbon atom, or a ring nitrogen atom if the ring is not thereby quaternised;
AR2a is a partially hydrogenated version of AR2 (i.e. AR2 systems retaining some, but not the full, degree of unsaturation), linked via a ring carbon atom or linked via a ring nitrogen atom if the ring is not thereby quaternised;
AR2b is a fully hydrogenated version of AR2 (i.e. AR2 systems having no unsaturation), linked via a ring carbon atom or linked via a ring nitrogen atom;
AR3 is an optionally substituted 8-, 9- or 10-membered, fully unsaturated (i.e with the maximum degree of unsaturation) bicyclic heteroaryl ring containing up to four heteroatoms independently selected from O, N and S (but not containing any O—O, O—S or S—S bonds), and linked via a ring carbon atom in either of the rings comprising the bicyclic system;
AR3a is a partially hydrogenated version of AR3 (i.e. AR3 systems retaining some, but not the full, degree of unsaturation), linked via a ring carbon atom, or linked via a ring nitrogen atom if the ring is not thereby quaternised, in either of the rings comprising the bicyclic system;
AR3b is a fully hydrogenated version of AR3 (i.e. AR3 systems having no unsaturation), linked via a ring carbon atom, or linked via a ring nitrogen atom, in either of the rings comprising the bicyclic system;
AR4 is an optionally substituted 13- or 14-membered, fully unsaturated (i.e with the maximum degree of unsaturation) tricyclic heteroaryl ring containing up to four heteroatoms independently selected from O, N and S (but not containing any O—O, O—S or S—S bonds), and linked via a ring carbon atom in any of the rings comprising the tricyclic system;
AR4a is a partially hydrogenated version of AR4 (i.e. AR4 systems retaining some, but not the full, degree of unsaturation), linked via a ring carbon atom, or linked via a ring nitrogen atom if the ring is not thereby quaternised, in any of the rings comprising the tricyclic system;
CY1 is an optionally substituted cyclobutyl, cyclopentyl or cyclohexyl ring;
CY2 is an optionally substituted cyclopentenyl or cyclohexenyl ring;
wherein; optional substituents on AR1, AR2, AR2a, AR2b, AR3, AR3a, AR3b, AR4, AR4a, CY1 and CY2 are (on an available carbon atom) up to three substituents independently selected from (1-4C)alkyl (optionally substituted by substituents selected independently from hydroxy, trifluoromethyl, (1-4C)alkyl S(O)_{q}— (q is 0, 1 or 2), (1-4C)alkoxy, (1-4C)alkoxycarbonyl, cyano, nitro, (1-4C)alkanoylamino, —CONRvRw or —NRvRw}, trifluoromethyl, hydroxy, halo, nitro, cyano, thiol, (1-4C)alkoxy, (1-4C)alkanoyloxy, dimethylaminomethyleneaminocarbonyl, di(N-(1-4C)alkyl)aminomethylimino, carboxy, (1-4C)alkoxycarbonyl, (1-4C)alkanoyl, (1-4C)alkylSO₂amino, (2-4C)alkenyl {optionally substituted by carboxy or (1-4C)alkoxycarbonyl}, (2-4C)alkynyl, (1-4C)alkanoylamino, oxo (═O), thioxo (═S), (1-4C)alkanoylamino {the (1-4C)alkanoyl group being optionally substituted by hydroxy}, (1-4C)alkyl S(O)_{q}— (q is 0, 1 or 2) {the (1-4C)alkyl group being optionally substituted by one or more groups independently selected from cyano, hydroxy and (1-4C)alkoxy}, —CONRvRw or —NRvRw [wherein Rv is hydrogen or (1-4C)alkyl; Rw is hydrogen or (1-4C)alkyl];
and further optional substituents on AR1, AR2, AR2a, AR2b, AR3, AR3a, AR3b, AR4, AR4a, CY1 and CY2 (on an available carbon atom), and also on alkyl groups (unless indicated otherwise) are up to three substituents independently selected from trifluoromethoxy, benzoylamino, benzoyl, phenyl {optionally substituted by up to three substituents independently selected from halo, (1-4C)alkoxy or cyano}, furan, pyrrole, pyrazole, imidazole, triazole, pyrimidine, pyridazine, pyridine, isoxazole, oxazole, isothiazole, thiazole, thiophene, hydroxyimino(1-4C)alkyl, (1-4C)alkoxyimino(1-4C)alkyl, halo-(1-4C)alkyl, (1-4C)alkanesulfonamido, —SO₂NRvRw [wherein Rv is hydrogen or (1-4C)alkyl; Rw is hydrogen or (1-4C)alkyl]; and
optional substituents on AR2, AR2a, AR2b, AR3, AR3a, AR3b, AR4 and AR4a are (on an available nitrogen atom, where such substitution does not result in quaternization) (1-4C)alkyl, (1-4C)alkanoyl {wherein the (1-4C)alkyl and (1-4C)alkanoyl groups are optionally substituted by (preferably one) substituents independently selected from cyano, hydroxy, nitro, trifluoromethyl, (1-4C)alkyl S(O)_{q}— (q is 0, 1 or 2), (1-4C)alkoxy, (1-4C)alkoxycarbonyl, (1-4C)alkanoylamino, —CONRvRw or —NRvRw [wherein Rv is hydrogen or (1-4C)alkyl; Rw is hydrogen or (1-4C)alkyl]}, (2-4C)alkenyl, (2-4C)alkynyl, (1-4C)alkoxycarbonyl or oxo (to form an N-oxide).

In another aspect, the invention relates to compounds of formula (1) as hereinabove defined or to a pharmaceutically acceptable salt.

In another aspect, the invention relates to compounds of formula (1) as hereinabove defined or to a pro-drug thereof. Suitable examples of pro-drugs of compounds of formula (1) are in-vivo hydrolysable esters of compounds of formula (1). Therefore in another aspect, the invention relates to compounds of formula (1) as hereinabove defined or to an in-vivo hydrolysable ester thereof.

Where optional substituents are chosen from “0, 1, 2 or 3” groups it is to be understood that this definition includes all substituents being chosen from one of the specified groups or the substituents being chosen from two or more of the specified groups. An analogous convention applies to substituents chose from “0, 1 or 2” groups and “1 or 2” groups.

In this specification the term ‘alkyl’ includes straight chained and branched structures. For example, (1-4C)alkyl includes propyl and isopropyl. However, references to individual alkyl groups such as “propyl” are specific for the straight chained version only, and references to individual branched chain alkyl groups such as “isopropyl” are specific for the branched chain version only. In this specification, the terms ‘alkenyl’ and ‘cycloalkenyl’ include all positional and geometrical isomers. In this specification, the term ‘aryl’ is an unsubstituted carbocyclic aromatic group, in particular phenyl, 1- and 2-naphthyl.

For the avoidance of doubt, reference to a carbon atom in HET-1 being substituted by an oxo or thioxo group means replacement of a CH₂ by C═O or C═S respectively.

Within this specification composite terms are used to describe groups comprising more that one functionality such as (1-4C)alkoxy-(1-4C)alkoxy-(1-4C)alkyl. Such terms are to be interpreted in accordance with the meaning which is understood by a person skilled in the art for each component part. For example (1-4C)alkoxy-(1-4C)alkoxy-(1-4C)alkyl includes methoxymethoxymethyl, ethoxymethoxypropyl and propxyethoxymethyl.

It will be understood that where a group is defined such that is optionally substituted by more than one substituent, then substitution is such that chemically stable compounds are formed. For example, a trifluoromethyl group may be allowed but not a trihydroxymethyl group. This convention is applied wherever optional substituents are defined.

The term “a C5-C6 heteroaromatic ring” means a 5- or 6-membered aryl ring wherein (unless stated otherwise) 1, 2 or 3 of the ring atoms are selected from nitrogen, oxygen and sulfur. Unless stated otherwise, such rings are fully aromatic. Particular examples of 5- or 6-membered heteroaryl ring systems are furan, pyrrole, pyrazole, imidazole, triazole, pyrimidine, pyridazine, pyridine, isoxazole, oxazole, isothiazole, thiazole and thiophene.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

Examples of (1-4C)alkyl and (1-5C)alkyl include methyl, ethyl, propyl, isopropyl and t-butyl; examples of (1-6C)alkyl include methyl, ethyl, propyl, isopropyl, t-butyl, pentyl and hexyl; examples of (1-10C)alkyl include methyl, ethyl, propyl, isopropyl, pentyl, hexyl, heptyl, octyl and nonyl; examples of (1-4C)alkanoylamino-(1-4C)alkyl include formamidomethyl, acetamidomethyl and acetamidoethyl; examples of hydroxy(1-4C)alkyl and hydroxy(1-6C)alkyl include hydroxymethyl, 1-hydroxyethyl, 2-hydroxyethyl and 3-hydroxypropyl; examples of (1-4C)alkoxycarbonyl include methoxycarbonyl, ethoxycarbonyl and propoxycarbonyl; examples of (1-4C)alkoxy-(1-4C)alkoxycarbonyl include methoxymethoxycarbonyl, methoxyethoxycarbonyl and propoxymethoxycarbonyl; examples of (1-4C)alkoxy-(1-4C)alkoxy-(1-4C)alkoxycarbonyl include methoxymethoxymethoxycarbonyl, methoxyethoxymethoxycarbonyl and propoxyethoxymethoxycarbonyl; examples of 2-((1-4C)alkoxycarbonyl)ethenyl include 2-(methoxycarbonyl)ethenyl and 2-(ethoxycarbonyl)ethenyl; examples of 2-cyano-2-((1-4C)alkyl)ethenyl include 2-cyano-2-methylethenyl and 2-cyano-2-ethylethenyl; examples of 2-nitro-2-((1-4C)alkyl)ethenyl include 2-nitro-2-methylethenyl and 2-nitro-2-ethylethenyl; examples of 2-((1-4C)alkylaminocarbonyl)ethenyl include 2-(methylaminocarbonyl)ethenyl and 2-(ethylaminocarbonyl)ethenyl; examples of (2-4C)alkenyl include allyl and vinyl; examples of (2-4C)alkenyloxy include allyloxy and vinyloxy; examples of (2-4C)alkynyl include ethynyl and 2-propynyl; examples of (2-4C)alkynyloxy include ethynyloxy and 2-propynyloxy; examples of (1-4C)alkanoyl include formyl, acetyl and propionyl; examples of (1-4C)alkylcarbonyl include acetyl and propionyl; examples of (1-4C)alkoxy include methoxy, ethoxy and propoxy; examples of (1-6C)alkoxy and (1-10C)alkoxy include methoxy, ethoxy, propoxy and pentoxy; examples of (1-4C)alkylthio include methylthio and ethylthio; examples of (1-4C)alkylamino include methylamino, ethylamino and propylamino; examples of (2-4C)alkenylamino include vinylamino and allylamino; examples of hydroxy(1-4C)alkylamino include 2-hydroxyethylamino, 2-hydroxypropylamino and 3-hydroxypropylamino; examples of di-((1-4C)alkyl)amino include dimethylamino, N-ethyl-N-methylamino, diethylamino, N-methyl-N-propylamino and dipropylamino; examples of halo groups include fluoro, chloro and bromo; examples of (1-4C)alkylsulfonyl include methylsulfonyl and ethylsulfonyl; examples of (1-4C)alkoxy-(1-4C)alkoxy and (1-6C)alkoxy-(1-6C)alkoxy include methoxymethoxy, 2-methoxyethoxy, 2-ethoxyethoxy and 3-methoxypropoxy; examples of (1-4C)alkoxy-(1-4C)alkoxy-(1-4C)alkoxy include 2-(methoxymethoxy)ethoxy, 2-(2-methoxyethoxy)ethoxy; 3-(2-methoxyethoxy)propoxy and 2-(2-ethoxyethoxy)ethoxy; examples of (1-4C)alkylS(O)₂amino include methylsulfonylamino and ethylsulfonylamino; examples of (1-4C)alkanoylamino and (1-6C)alkanoylamino include formamido, acetamido and propionylamino; examples of (1-4C)alkoxycarbonylamino include methoxycarbonylamino and ethoxycarbonylamino; examples of N-(1-4C)alkyl-N-(1-6C)alkanoylamino include N-methylacetamido, N-ethylacetamido and N-methylpropionamido; examples of (1-4C)alkylS(O)ₚNH— wherein p is 1 or 2 include methylsulfinylamino, methylsulfonylamino, ethylsulfinylamino and ethylsulfonylamino; examples of (1-4C)alkylS(O)ₚ((1-4C)alkyl)N— wherein p is 1 or 2 include methylsulfinylmethylamino, methylsulfonylmethylamino, 2-(ethylsulfinyl)ethylamino and 2-(ethylsulfonyl)ethylamino; examples of fluoro(1-4C)alkylS(O)ₚNH— wherein p is 1 or 2 include trifluoromethylsulfinylamino and trifluoromethylsulfonylamino; examples of fluoro(1-4C)alkylS(O)ₚ((1-4C)alkyl)NH— wherein p is 1 or 2 include trifluoromethylsulfinylmethylamino and trifluoromethylsulfonylmethylamino examples of (1-4C)alkoxy(hydroxy)phosphoryl include methoxy(hydroxy)phosphoryl and ethoxy(hydroxy)phosphoryl; examples of di-(1-4C)alkoxyphosphoryl include di-methoxyphosphoryl, di-ethoxyphosphoryl and ethoxy(methoxy)phosphoryl; examples of (1-4C)alkylS(O)_{q}— wherein q is 0, 1 or 2, and —S(O)n(1-4C)alkyl (wherein n=1 or 2), include methylthio, ethylthio, methylsulfinyl, ethylsulfinyl, methylsulfonyl and ethylsulfonyl; examples of phenylS(O)_{q} and naphthylS(O)_{q}— wherein q is 0, 1 or 2 are phenylthio, phenylsulfinyl, phenylsulfonyl and naphthylthio, naphthylsulfinyl and naphthylsulfonyl respectively; examples of benzyloxy-(1-4C)alkyl include benzyloxymethyl and benzyloxyethyl; examples of a (3-4C)alkylene chain are trimethylene or tetramethylene; examples of (1-6C)alkoxy-(1-6C)alkyl include methoxymethyl, ethoxymethyl and 2-methoxyethyl; examples of hydroxy-(2-6C)alkoxy include 2-hydroxyethoxy and 3-hydroxypropoxy; examples of (1-4C)alkylamino-(2-6C)alkoxy include 2-methylaminoethoxy and 2-ethylaminoethoxy; examples of di-(1-4C)alkylamino-(2-6C)alkoxy include 2-dimethylaminoethoxy and 2-diethylaminoethoxy; examples of phenyl(1-4C)alkyl include benzyl and phenethyl; examples of (1-4C)alkylcarbamoyl include methylcarbamoyl and ethylcarbamoyl; examples of di((1-4C)alkyl)carbamoyl include di(methyl)carbamoyl and di(ethyl)carbamoyl; examples of hydroxyimino(1-4C)alkyl include hydroxyiminomethyl, 2-(hydroxyimino)ethyl and 1-(hydroxyimino)ethyl; examples of (1-4C)alkoxyimino-(1-4C)alkyl include methoxyiminomethyl, ethoxyiminomethyl, 1-(methoxyimino)ethyl and 2-(methoxyimino)ethyl; examples of halo(1-4C)alkyl include, halomethyl, 1-haloethyl, 2-haloethyl, and 3-halopropyl; examples of nitro(1-4C)alkyl include nitromethyl, 1-nitroethyl, 2-nitroethyl and 3-nitropropyl; examples of amino(1-4C)alkyl include aminomethyl, 1-aminoethyl, 2-aminoethyl and 3-aminopropyl; examples of cyano(1-4C)alkyl include cyanomethyl, 1-cyanoethyl, 2-cyanoethyl and 3-cyanopropyl; examples of (1-4C)alkanesulfonamido include methanesulfonamido and ethanesulfonamido; examples of (1-4C)alkylaminosulfonyl include methylaminosulfonyl and ethylaminosulfonyl; examples of di-(1-4C)alkylaminosulfonyl include dimethylaminosulfonyl, diethylaminosulfonyl and N-methyl-N-ethylaminosulfonyl; examples of (1-4C)alkanesulfonyloxy include methylsulfonyloxy, ethylsulfonyloxy and propylsulfonyloxy; examples of (1-4C)alkanoyloxy include acetoxy and propionyloxy; examples of (1-4C)alkylaminocarbonyl include methylaminocarbonyl and ethylaminocarbonyl; examples of di((1-4C)alkyl)aminocarbonyl include dimethylaminocarbonyl and diethylaminocarbonyl; examples of (3-6C)cycloalkyl include cyclopropyl, cyclobutyl, cyclopentyl and cyclohexyl; examples of (3-6C)cycloalkenyl include cyclopropenyl, cyclobutenyl, cyclopentenyl and cyclohexenyl; examples of (4-7C)cycloalkyl include cyclobutyl, cyclopentyl and cyclohexyl; examples of di(N-(1-4C)alkyl)aminomethylimino include dimethylaminomethylimino and diethylaminomethylimino.

Particular values for AR2 include, for example, for those AR2 containing one heteroatom, furan, pyrrole, thiophene; for those AR2 containing one to four N atoms, pyrazole, imidazole, pyridine, pyrimidine, pyrazine, pyridazine, 1,2,3- & 1,2,4-triazole and tetrazole; for those AR2 containing one N and one O atom, oxazole, isoxazole and oxazine; for those AR2 containing one N and one S atom, thiazole and isothiazole; for those AR2 containing two N atoms and one S atom, 1,2,4- and 1,3,4-thiadiazole.

Particular examples of AR2a include, for example, dihydropytrole (especially 2,5-dihydropyrrol-4-yl) and tetrahydropyridine (especially 1,2,5,6-tetrahydropyrid-4-yl).

Particular examples of AR2b include, for example, tetrahydrofuran, pyrrolidine, morpholine (preferably morpholino), thiomorpholine (preferably thiomorpholino), piperazine (preferably piperazino), imidazoline and piperidine, 1,3-dioxolan-4-yl, 1,3-dioxan-4-yl, 1,3-dioxan-5-yl and 1,4-dioxan-2-yl.

Particular values for AR3 include, for example, bicyclic benzo-fused systems containing a 5- or 6-membered heteroaryl ring containing one nitrogen atom and optionally 1-3 further heteroatoms chosen from oxygen, sulfur and nitrogen. Specific examples of such ring systems include, for example, indole, benzofuran, benzothiophene, benzimidazole, benzothiazole, benzisothiazole, benzoxazole, benzisoxazole, quinoline, quinoxaline, quinazoline, phthalazine and cinnoline.

Other particular examples of AR3 include 5/5-, 5/6 and 6/6 bicyclic ring systems containing heteroatoms in both of the rings. Specific examples of such ring systems include, for example, purine and naphthyridine.

Further particular examples of AR3 include bicyclic heteroaryl ring systems with at least one bridgehead nitrogen and optionally a further 1-3 heteroatoms chosen from oxygen, sulfur and nitrogen. Specific examples of such ring systems include, for example, 3H-pyrrolo[1,2-a]pyrrole, pyrrolo[2,1-b]thiazole, 1H-imidazo[1,2-a]pyrrole, 1H-imidazo[1,2-a]imidazole, 1H,3H-pyrrolo[1,2-c]oxazole, 1H-imidazo[1,5-a]pyrrole, pyrrolo[1,2-b]isoxazole, imidazo[5,1-b]thiazole, imidazo[2,1-b]thiazole, indolizine, imidazo[1,2-a]pyridine, imidazo[1,5-a]pyridine, pyrazolo[1,5-a]pyridine, pyrrolo[1,2-b]pyridazine, pyrrolo[1,2-c]pyrimidine, pyrrolo[1,2-a]pyrazine, pyrrolo[1,2-a]pyrimidine, pyrido[2,1-c]-s-triazole, s-triazole[1,5-a]pyridine, imidazo[1,2-c]pyrimidine, imidazo[1,2-a]pyrazine, imidazo[1,2-a]pyrimidine, imidazo[1,5-a]pyrazine, imidazo[1,5-a]pyrimidine, imidazo[1,2-b]-pyridazine, s-triazolo[4,3-a]pyrimidine, imidazo[5,1-b]oxazole and imidazo[2,1-b]oxazole. Other specific examples of such ring systems include, for example, [1H]-pyrrolo[2,1-c]oxazine, [3H]-oxazolo[3,4a]pyridine, [6H]-pyrrolo[2,1-c]oxazine and pyrido[2,1-c][1,4]oxazine. Other specific examples of 5/5-bicyclic ring systems are imidazooxazole or imidazothiazole, in particular imidazo[5,1-b]thiazole, imidazo[2,1-b]thiazole, imidazo[5,1-b]oxazole or imidazo[2,1-b]oxazole.

Particular examples of AR3a and AR3b include, for example, indoline, 1,3,4,6,9,9a-hexahydropyrido[2,1c][1,4]oxazin-8-yl, 1,2,3,5,8,8a-hexahydroimidazo[1,5a]pyridin-7-yl, 1,5,8,8a-tetrahydrooxazolo[3,4a]pyzidin-7-yl, 1,5,6,7,8,8a-hexahydrooxazolo[3,4a]pyridin-7-yl, (7aS)[3H,5H]-1,7a-dihydropyrrolo[1,2c]oxazol-6-yl, (7aS)[5H]-1,2,3,7a-tetrahydropyrrolo[1,2c]imidazol-6-yl, (7aR)[3H,5H]-1,7a-dihydropyrrolo[1,2c]oxazol-6-yl, [3H,5H]-pyrrolo[1,2-c]oxazol-6-yl, [5H]-2,3-dihydropyrrolo[1,2-c]imidazol-6-yl, [3H,5H]-pyrrolo[1,2-c]thiazol-6-yl, [3H,5H]-1,7a-dihydropyrrolo[1,2-c]thiazol-6-yl, [5H]-pyrrolo[1,2-c]imidazol-6-yl, [1H]-3,4,8,8a-tetrahydropyrrolo[2,1-c]oxazin-7-yl, [311]-1,5,8,8a-tetrahydrooxazolo-[3,4-a]pyrid-7-yl, [3H]-5,8-dihydroxazolo[3,4-a]pyrid-7-yl and 5,8-dihydroimidazo-[1,5-a]pyrid-7-yl.

Particular values for AR4 include, for example, pyrrolo[a]quinoline, 2,3-pyrroloisoquinoline, pyrrolo[a]isoquinoline, 1H-pyrrolo[1,2-a]benzimidazole, 9H-imidazo[1,2-a]indole, 5H-imidazo[2,1-a]isoindole, 1H-imidazo[3,4-a]indole, imidazo[1,2-a]quinoline, imidazo[2,1-a]isoquinolne, imidazo[1,5-a]quinoline and imidazo[5,1-a]isoquinoline.

The nomenclature used is that found in, for example, “Heterocyclic Compounds (Systems with bridgehead nitrogen), W. L. Mosby (Interscience Publishers Inc., New York), 1961, Parts 1 and 2.

Where optional substituents are listed such substitution is preferably not geminal disubstitution unless stated otherwise. If not stated elsewhere, suitable optional substituents for a particular group are those as stated for similar groups herein.

Preferable optional substituents on Ar2b as 1,3-dioxolan-4-yl, 1,3-dioxan-4-yl, 1,3-dioxan-5-yl or 1,4-dioxan-2-yl are mono- or disubstitution by substituents independently selected from (1-4C)alkyl (including geminal disubstitution), (1-4C)alkoxy, (1-4C)alkylthio, acetamido, (1-4C)alkanoyl, cyano, trifluoromethyl and phenyl].

Preferable optional substituents on CY1 & CY2 are mono- or disubstitution by substituents independently selected from (1-4C)alkyl (including geminal disubstitution), hydroxy, (1-4C)alkoxy, (1-4C)alkylthio, acetamido, (1-4C)alkanoyl, cyano, and trifluoromethyl.

Suitable pharmaceutically-acceptable salts include acid addition salts such as methanesulfonate, fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

The compounds of the invention may be administered in the form of a pro-drug which is broken down in the human or animal body to give a compound of the invention. A prodrug may be used to alter or improve the physical and/or pharmacokinetic profile of the parent compound and can be formed when the parent compound contains a suitable group or substituent which can be derivatised to form a prodrug. Examples of prodrugs include in-vivo hydrolysable esters of a compound of the invention or a pharmaceutically-acceptable salt thereof.

Various forms of prodrugs are known in the art, for examples see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

Suitable pro-drugs for pyridine or triazole derivatives include acyloxymethyl pyridinium or triazolium salts eg halides; for example a pro-drug such as: 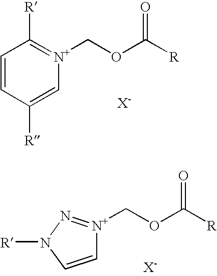
(Ref: T. Yamazaki et al. 42^{nd} Interscience Conference on Antimicrobial Agents and Chemotherapy, San Diego, 2002; Abstract F820).

Suitable pro-drugs of hydroxyl groups are acyl esters of acetal-carbonate esters of formula RCOOC(R,R′)OCO—, where R is (1-4C)alkyl and R′ is (1-4C)alkyl or H. Further suitable prodrugs are carbonate and carabamate esters RCOO— and RNHCOO—.

An in-vivo hydrolysable ester of a compound of the invention or a pharmaceutically-acceptable salt thereof containing a carboxy or hydroxy group is, for example, a pharmaceutically-acceptable ester which is hydrolysed in the human or animal body to produce the parent alcohol.

Suitable pharmaceutically-acceptable esters for carboxy include (1-6C)alkoxymethyl esters for example methoxymethyl, (1-6C)alkanoyloxymethyl esters for example pivaloyloxymethyl, phthalidyl esters, (3-8C)cycloalkoxycarbonyloxy(1-6C)alkyl esters for example 1-cyclohexylcarbonyloxyethyl; 1,3-dioxolan-2-onylmethyl esters for example 5-methyl-1,3-dioxolan-2-ylmethyl; and (1-6C)alkoxycarbonyloxyethyl esters for example 1-methoxycarbonyloxyethyl and may be formed at any carboxy group in the compounds of this invention.

An in-vivo hydrolysable ester of a compound of the invention or a pharmaceutically-acceptable salt thereof containing a hydroxy group or groups includes inorganic esters such as phosphate esters (including phosphoramidic cyclic esters) and x-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxymethoxy. A selection of in-vivo hydrolysable ester forming groups for hydroxy include (1-10C)alkanoyl, benzoyl, phenylacetyl and substituted benzoyl and phenylacetyl, (1-10C)alkoxycarbonyl (to give alkyl carbonate esters), di-(1-4C)alkylcarbamoyl and N-(di-(1-4C)alkylaminoethyl)-N-(1-4C)alkylcarbamoyl (to give carbamates), di-(1-4C)alkylaminoacetyl, carboxy(2-5C)alkylcarbonyl and carboxyacetyl. Examples of ring substituents on phenylacetyl and benzoyl include chloromethyl or aminomethyl, (1-4C)alkylaminomethyl and di-((1-4C)alkyl)aminomethyl, and morpholino or piperazino linked from a ring nitrogen atom via a methylene linking group to the 3- or 4-position of the benzoyl ring. Other interesting in-vivo hydrolysable esters include, for example, R^{A}C(O)O(1-6C)alkyl-CO— (wherein R^{A} is for example, optionally substituted benzyloxy-(1-4C)alkyl, or optionally substituted phenyl; suitable substituents on a phenyl group in such esters include, for example, 4-(1-4C)piperazino-(1-4C)alkyl, piperazino-(1-4C)alkyl and morpholino-(1-4C)alkyl.

Suitable in-vivo hydrolysable esters of a compound of the formula (I) are described as follows. For example, a 1,2-diol may be cyclised to form a cyclic ester of formula (PD1) or a pyrophosphate of formula (PD2), and a 1,3-diol may be cyclised to form a cyclic ester of the formula (PD3):

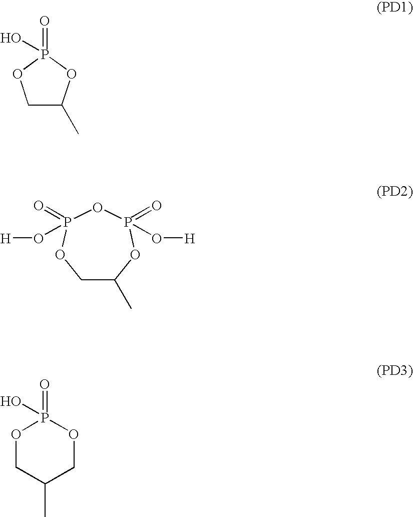

Esters of compounds of formula (I) wherein the HO— function/s in (PD1), (PD2) and (PD3) are protected by (1-4C)alkyl, phenyl or benzyl are useful intermediates for the preparation of such pro-drugs.

Further in-vivo hydrolysable esters include phosphoramidic esters, and also compounds of invention in which any free hydroxy group independently forms a phosphoryl (npd is 1) or phosphiryl (npd is 0) ester of the formula (PD4):

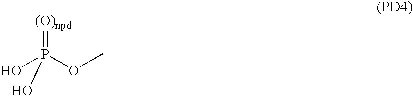

For the avoidance of doubt, phosphono is —P(O)(OH)₂; (1-4C)alkoxy(hydroxy)-phosphoryl is a mono-(1-4C)alkoxy derivative of —O—P(O)(OH)₂; and di-(1-4C)alkoxyphosphoryl is a di-(1-4C)alkoxy derivative of —O—P(O)(OH)₂.

Useful intermediates for the preparation of such esters include compounds containing a group/s of formula (PD4) in which either or both of the —OH groups in (PD1) is independently protected by (1-4C)alkyl (such compounds also being interesting compounds in their own right), phenyl or phenyl-(1-4C)alkyl (such phenyl groups being optionally substituted by 1 or 2 groups independently selected from (1-4C)alkyl, nitro, halo and (1-4C)alkoxy).

Thus, prodrugs containing groups such as (PD1), (PD2), (PD3) and (PD4) may be prepared by reaction of a compound of invention containing suitable hydroxy group/s with a suitably protected phosphorylating agent (for example, containing a chloro or dialkylamino leaving group), followed by oxidation (if necessary) and deprotection.

Other suitable prodrugs include phosphonooxymethyl ethers and their salts, for example a prodrug of R—OH such as:

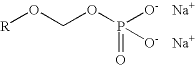

When a compound of invention contains a number of free hydroxy group, those groups not being converted into a prodrug functionality may be protected (for example, using a t-butyl-dimethylsilyl group), and later deprotected. Also, enzymatic methods may be used to selectively phosphorylate or dephosphorylate alcohol functionalities.

Where pharmaceutically-acceptable salts of an in-vivo hydrolysable ester may be formed this is achieved by conventional techniques. Thus, for example, compounds containing a group of formula (PD1), (PD2), (PD3)and/or (PD4) may ionise (partially or fully) to form salts with an appropriate number of counter-ions. Thus, by way of example, if an in-vivo hydrolysable ester prodrug of a compound of invention contains two (PD4) groups, there are four HO—P— functionalities present in the overall molecule, each of which may form an appropriate salt (i.e. the overall molecule may form, for example, a mono-, di-, tri- or tetra-sodium salt).

The compounds of the present invention have a chiral centre at the C-5 positions of the oxazolidinone ring. The pharmaceutically active diastereomer is of the formula (Ia): 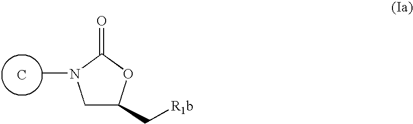
which is generally the (5R) configuration, depending on the nature of R₁b and C.

The present invention includes pure diastereomers or mixtures of diastereomers, for example a racemic mixture. If a mixture of enantiomers is used, a larger amount (depending upon the ratio of the enantiomers) will be required to achieve the same effect as the same weight of the pharmaceutically active enantiomer.

Furthermore, some compounds of the invention may have other chiral centres, for example on substituents on group C. It is to be understood that the invention encompasses all such optical and diastereoisomers, and racemic mixtures, that possess antibacterial activity. It is well known in the art how to prepare optically-active forms (for example by resolution of the racemic form by recrystallisation techniques, by chiral synthesis, by enzymatic resolution, by biotransformation or by chromatographic separation) and how to determine antibacterial activity as described hereinafter.

The invention relates to all tautomeric forms of the compounds of the invention that possess antibacterial activity.

It is also to be understood that certain compounds of the invention can exist in solvated as well as unsolvated forms such as, for example, hydrated forms. It is to be understood that the invention encompasses all such solvated forms which possess antibacterial activity.

It is also to be understood that certain compounds of the invention may exhibit polymorphism, and that the invention encompasses all such forms which possess antibacterial activity.

As stated before, we have discovered a range of compounds that have good activity against a broad range of Gram-positive pathogens including organisms known to be resistant to most commonly used antibiotics, together with activity against fastidious Gram negative pathogens such as H.influenzae, M.catarrhalis, Mycoplasma and Chlamydia strains. The following compounds possess preferred pharmaceutical and/or physical and/or pharmacokinetic properties.

In one embodiment of the invention are provided compounds of formula (I), in an alternative embodiment are provided pharmaceutically-acceptable salts of compounds of formula (I), in a further alternative embodiment are provided in-vivo hydrolysable esters of compounds of formula (I), and in a further alternative embodiment are provided pharmaceutically-acceptable salts of in-vivo hydrolysable esters of compounds of formula (I).

In one aspect, an in-vivo hydrolysable ester of a compound of the formula (I) is a phosphoryl ester (as defined by formula PD4) with npd as 1).

Compounds of the formula (I), or a pharmaceutically-acceptable salt or an in-vivo hydrolysable ester thereof, wherein C is selected from group D or group E represent separate and independent aspects of the invention.

Particularly preferred compounds of the invention comprise a compound of the invention, or a pharmaceutically-acceptable salt or an in-vivo hydrolysable ester thereof, wherein the substituents R₁a, R₁b, R₂a, R₂b, R₃a, R₆a and R₆b and other substituents mentioned above have values disclosed hereinbefore, or any of the following values (which may be used where appropriate with any of the definitions and embodiments disclosed hereinbefore or hereinafter):

In one embodiment are provided compounds of the formula (I) or pharmaceutically acceptable salt or in-vivo hydrolysable ester thereof wherein group C is group D.

In another embodiment are provided compounds of the formula (I) or pharmaceutically acceptable salt or in-vivo hydrolysable ester thereof wherein group C is group E.

In one aspect R₂a and R₆a are hydrogen.

In one aspect one R₂b and R₆b is fluoro and the other is hydrogen. In another aspect both one R₂b and R₆b are fluoro. In a further aspect R₂b is fluoro and R₆b is selected from Cl, CF₃, Me, Et, OMe and SMe.

In one aspect one of R₂b and R₆b is chloro and other hydrogen.

In another aspect one of R₂b and R₆b is CF₃ and the other hydrogen.

In another aspect one of R₂b and R₆b is Me and the other hydrogen.

In another aspect one of R₂b and R₆b is Et and the other hydrogen.

In another aspect one of R₂b and R₆b is OMe and the other hydrogen.

In another aspect one of R₂b and R₆b is SMe and the other hydrogen.

In one aspect R₃a is selected from H, (1-4C)alkyl, cyano, Br, F, Cl, OH, (1-4C)alkoxy, —S(1-4C)alkyl, amino, nitro and —CHO. In a further aspect R₃a is selected from H, Cl, Br, F, Me, Et, OMe and SMe.

In one aspect, HET1 is HET 1A. Conveniently HET-1A is isoxazolyl, 1,2,5-thiadiazolyl or isothiazolyl. More conveniently HET-1A is isoxazolyl.

In another aspect, HET1 is HET1B. Conveniently HET1B is pyrimidine, pyridazine, pyrazine, 1,2,3-triazine, 1,2,4-triazine and 1,3,5-triazine.

In one aspect RT is selected from a substituent from the groups RTa1 to RTb2, wherein:

(RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano and nitro;

(RTa2) (1-4C)alkylamino, di-(1-4C)alkylamino and (2-4C)alkenylamino;

(RTb1) a (1-4C)alkyl group which is optionally substituted by one substituent selected from hydroxy, (1-4C)alkoxy, (1-4C)alkylthio, cyano and azido;

(RTb2) a (1-4C)alkyl group which is optionally substituted by one substituent selected from (2-4C)alkenyloxy, (3-6C)cycloalkyl and (3-6C)cycloalkenyl;

and wherein at each occurrence of an RT substituent containing an alkyl, alkenyl, alkynyl, cycloalkyl or cycloalkenyl moiety in (RTa1) or (RTa2), or (RTb1) or (RTb2) each such moiety is optionally substituted on an available carbon atom with one, two, three or more substituents independently selected from F, Cl, Br, OH and CN.

In another aspect RT is preferably selected from a substituent from the groups RTa1 and RTb1, wherein:

(RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano, and nitro;

(RTb1) a (1-4C)alkyl group which is optionally substituted by one substituent selected from hydroxy, (1-4C)alkoxy, (1-4C)alkylthio, cyano and azido;

and wherein at each occurrence of an RT substituent containing an alkyl, alkenyl, alkynyl, cycloalkyl or cycloalkenyl moiety in (RTa1) or (RTb1) each such moiety is optionally substituted on an available carbon atom with one, two, three or more substituents independently selected from F, Cl, Br, and CN.

In a further aspect RT is most preferably
(a) hydrogen; or
(b) halogen, in particular fluorine, chlorine, or bromine; or
(c) cyano; or
(d) (1-4C)alkyl, in particular methyl; or
(e) monosubstituted (1-4C)alkyl, in particular fluoromethyl, choromethyl, bromomethyl, cyanomethyl, azidomethyl, hydroxymethyl; or
(f) disubstituted (1-4C)alkyl, for example difluoromethyl, or
(g) trisubstituted (1-4C)alkyl, for example trifluoromethyl.

In one aspect R₄ is selected from R₄a. In another aspect R₄ is selected from R₄b.

In one aspect R₄a is selected from (1-4C)alkyl, (1-4C)cycloalkyl, AR1, AR2, (1-4C)acyl, —CS(1-4C)alkyl, —C(═W)NRvRw [wherein W is O or S, Rv and Rw are independently H, or (1-4C)alkyl], —COO(1-4C)alkyl, —C═OAR1, —C═OAR2, —COOAR1, S(O)n(1-4C)alkyl (wherein n=1 or 2), —(O)pAR1, —S(O)pAR2 and —C(═S)O(1-4C)alkyl; wherein any (1-4C)alkyl chain may be optionally substituted by (1-4C)alkyl, cyano, hydroxy or halo; p=0, 1 or 2).

In a further aspect R₄a is selected from azido, —NR₇R₈, OR₁₀(1-4C)alkoxy, —(CH2)ₘ-R9 and —(C═O)₁—R6,

In one aspect HET-3 is selected from HET3-A, HET3-B, HET3-C, HET3-D and HET3-E.

In another aspect HET-3 is selected from HET3-F, HET3-G, HET3-H and HET3-I.

In another aspect HET-3 is selected from HET3-J, HET3-K, HET3-L, HET3-M, HET3-N, HET3-O, HET3-P, HET3-Q, HET3-R and HET3-S.

In a further aspect HET-3 is selected from HET3-J, HET3-L, HET3-M, HET3-N, HET3-P, HET3-Q, HET3-R and HET3-S.

In a further aspect HET-3 is selected from HET3-L and HET3-M.

In a further aspect HET-3 is selected from HET3-P and HET3-Q

In a further aspect HET-3 is selected from HET3-T, HET3-U, HET3-V, HET3-W, HET3-X and HET3-Y.

In a further aspect HET-3 is selected HET3-T, HET3-V, HET3-Y and HET-3-W.

In a further aspect HET-3 is selected HET3-V, and HET3-Y.

In a further aspect HET-3 is selected HET3-U, and HET3-X.

In a further aspect HET-3 is selected HET3-T, and HET3-W.

In a further aspect HET-3 is selected from HET3-Z, HET3-AA, HET3-AB, HET3-AC, HET3-AD, HET3-AE, HET3-AF, HET3-AG and HET3-AH.

When m=1, in one aspect R₁a is selected from R₁a1; in another aspect R₁a is selected from R₁a2; in a further aspect R₁a is selected from R₁a3, in a further aspect R₁a is selected from R₁a4 and in a further aspect R₁a is selected from R₁a5.

When m=2, in one aspect both groups R₁a are independently selected from the same group R₁a1 to R₁a5. In a further aspect when m=2, each R₁a is independently selected from different groups R₁a1 to R₁a5.

Conveniently, m is 1 or 2. In one aspect, preferably m is 1. In another aspect, preferably m is 2.

Particular values for R₁a when selected from R₁a1 are AR1 and AR2, more particularly AR2.

Particular values for R₁a when selected from R₁a2 are cyano and —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl (optionally substituted on a carbon not adjacent to the nitrogen), (3-6C)cycloalkyl, (1-4C)alkanoyl, —COO(1-4C)alkyl, —S(O)n(1-4C)alkyl (wherein n=1 or 2), —COOAR1, —CS(1-4C)alkyl and —C(═S)O(1-4C)alkyl; wherein any (1-4C)alkyl, (1-4C)alkanoyl and (3-6C)cycloalkyl is optionally substituted by cyano, hydroxy or halo]. More particular values for R₁a when selected from R₁a2 are cyano, formyl, —COO(1-4C)alkyl, —C(═O)NH₂, —(C—O)piperazine and —(C═O)morpholine.

Particular values for R₁a when selected from R₁a3 are (1-10C)alkyl {optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, (1-4C)alkylcarbonyl, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], and amino; and/or optionally substituted by one group selected from carboxy, cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, (1-4C)alkylamino, di((1-4C)alkyl)amino, (1-6C)alkanoylamino-, (1-4C)alkoxycarbonylamino-, N-(1-4C)alkyl-N-(1-6C)alkanoylamino-, —C(═W)NRvRw [wherein W is O, Rv and Rw are independently H, or (1-4C)alkyl and wherein Rv and Rw taken together with the amide nitrogen to which they are attached can form a morpholine, pyrrolidine, piperidine or piperazine ring; wherein when said ring is a piperazine ring, the ring may be optionally substituted on the additional nitrogen by a group selected from (1-4C)alkyl and (1-4C)alkanoyl], (1-4C)alkylS(O)_{q}—, (q is 0, 1 or 2), AR2, AR2-O—, AR2-NH—, and also AR2a, AR2b versions of AR2 containing groups}; wherein any (1-4C)alkyl and (1-4C)alkanoyl present in any substituent on R₁a3 may itself be substituted by one or two groups independently selected from cyano, hydroxy, halo, amino, (1-4C)alkylamino and di(1-4C)alkylamino, provided that such a substituent is not on a carbon adjacent to a heteroatom atom if present;

More particular values for R₁a when selected from R₁a3 are (1-10C)alkyl {optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], carboxy, amino, (1-4C)alkylamino, di(1-4C)alkylamino, (1-4C)alkylS(O)_{q} (preferably where q=2), AR2 and AR2b. More particular values for R₁a when selected from R₁a3 are (1-6C)alkyl substituted as hereinbefore described. Even more particular values for R₁a when selected from R₁a3 are (1-4C)alkyl substituted as hereinbefore described.

Particular values for substituents on a (1-10C)alkyl, (1-6C)alkyl or (1-4C)alkyl group comprising R₁a3 are hydroxy, (1-10C)alkoxy, (1-4C)alkoxy-(1-4C)alkoxy, (1-4C)alkoxy-(1-4C)alkoxy-(1-4C)alkoxy, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof] and carboxy. Preferably R₁a3 is a (1-4C)alkyl group substituted with 1 or 2 hydroxy groups.

Particular values for R₁a when selected from R₁a4 are R₁₄C(O)O(1-6C)alkyl- wherein R₁₄ is selected from AR1, AR2, AR2a, AR2b and (1-10C)alkyl (optionally substituted by one or more substituents independently selected from OH and di (1-4C)alkylamino. More particular vales for R₁₄ are AR2a, AR2b and (1-6C)alkyl substituted with hydroxy. More particular values for R₁₄ are AR2a, AR2b and (1-4C)alkyl substituted with hydroxy.

Particular values for R₁a when selected from R₁a5 are fluoro, chloro and hydroxy.

Particular values for other substituents (which may be used where appropriate with any of the definitions and embodiments disclosed hereinbefore or hereinafter) are:—

a) in one aspect R₇ and R₈ are independently H or (1-4C)alkyl

b) in a further aspect R₇ and R₈ taken together with the nitrogen to which they are attached form a 5-7 membered ring, optionally substituted as defined hereinbefore or hereinafter

c) preferably R₇ and R₈ taken together with the nitrogen to which they are attached form a pyrrolidinyl, piperidinyl, piperazinyl or morpholinyl ring

d) preferable optional subsituents on R₇ and R₈ as a pyrrolidinyl, piperidinyl, piperazinyl or morpholinyl ring are (1-4C)alkyl and (1-4C)alkanoyl, wherein the (1-4C)alkyl or (1-4C)alkanoyl group itself may be optionally substituted with one or two substituents selected from hydroxy, amino, (1-4C)alkylamino and di(1-4C)alkylamino

e) In one aspect R₉ is selected from R₉a, preferably selected from AR2, AR2a and AR2b

f) In another aspect R₉ is selected from R₉b, preferably selected from —C(═W)NRvRw, wherein wherein W is O, Rv and Rw are independently H, or (1-4C)alkyl and wherein Rv and Rw taken together with the amide nitrogen to which they are attached can form a morpholine, pyrrolidine, piperidine or piperazine ring; wherein when said ring is a piperazine ring, the ring may be optionally substituted on the additional nitrogen by a group selected from (1-4C)alkyl and (1-4C)alkanoyl, and wherein any (1-4C)alkyl and (1-4C)alkanoyl may itself be substituted by one or two groups independently selected from cyano, hydroxy, halo, amino, (1-4C)alkylamino and di(1-4C)alkylamino, provided that such a substituent is not on a carbon adjacent to a heteroatom atom if present

g) In a further aspect R₉ is selected from R₉c, wherein R₉c is (1-6C)alkyl {optionally substituted by one, two or three groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, (1-4C)alkylcarbonyl, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], and amino; and/or optionally substituted by one group selected from carboxy, cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, (1-4C)alkylamino, di((1-4C)alkyl)amino, (1-6C)alkanoylamino-, (1-4C)alkoxycarbonylamino-, N-(1-4C)alkyl-N-(1-6C)alkanoylamino-, —C(═W)NRvRw [wherein W is O, Rv and Rw are independently H, or (1-4C)alkyl and wherein Rv and Rw taken together with the amide nitrogen to which they are attached can form a morpholine, pyrrolidine, piperidine or piperazine ring; wherein when said ring is a piperazine ring, the ring may be optionally substituted on the additional nitrogen by a group selected from (1-4C)alkyl and (1-4C)alkanoyl], (1-4C)alkylS(O)_{q}— (q is 0, 1 or 2), AR2, AR2-O—, AR2-NH—, and also AR2a, AR2b versions of AR2 containing groups};

wherein any (1-4C)alkyl and (1-4C)alkanoyl present in any substituent on R₉c may itself be substituted by one or two groups independently selected from cyano, hydroxy, halo, amino, (1-4C)alkylamino and di(1-4C)alkylamino, provided that such a substituent is not on a carbon adjacent to a heteroatom atom if present.

h) In a further aspect R₉ is selected from R₉c, wherein R₉c is (1-6C)alkyl {optionally substituted by one, two or three groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], carboxy, amino, (1-4C)alkylamino, di(1-4C)alkylamino, (1-4C)alkylS(O)_{q} (preferably where q=2), AR2 and AR2b. More particular values for R₉c is (1-4C)alkyl, optionally substituted as hereinbefore described.

i) In a further aspect R₉ is selected from R₉d wherein R₉d is R₁₄C(O)O(1-6C)alkyl- and R₁₄ is selected from AR1, AR2, AR2a, AR2b and (1-10C)alkyl (optionally substituted by one or two substituents independently selected from OH and di (1-4C)alkylamino). Particular vales for R₁₄ are AR2a, AR2b and (1-6C)alkyl substituted with hydroxy. More particular values for R₁₄ are AR2a, AR2b and (1-4C)alkyl substituted with hydroxy.

j) Particular values for R₂₁ is R₁₄C(O)O(2-6C)alkyl-, wherein R₁₄ is preferably selected from AR1, AR2, AR2a, AR2b and (1-10C)alkyl (optionally substituted by one or two substituents independently selected from OH and di (1-4C)alkylamino.

k) Further particular values for R₂₁ are (2-10C)alkyl, optionally substituted other than on a carbon attached to the HET-3 ring nitrogen with one or two groups independently seleted from the optional substituents defined hereinbefore or hereinafter for R₁a3; further particular values for R₂₁ are optionally substituted (2-6C)alkyl, more particularly optionally substituted (2-4C)alkyl.

l) Particular substituents for a (2-6C)alkyl or (2-4C)alkyl group comprising R₂₁ are 1 or 2 substituents independently selected from hydroxy, (1-10C)alkoxy, (1-4C)alkoxy-(1-4C)alkoxy, (1-4C)alkoxy-(1-4C)alkoxy-(1-4C)alkoxy, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], carboxy, amino, (1-4C)alkylamino, di(1-4C)alkylamino, (1-4C)alkylS(O)_{q} (preferably where q=2), AR2 and AR2b

m) Further particular values for substituents on a (2-6C)alkyl or (2-4C)alkyl group comprising R₂₁ are 1 or 2 substituents independently selected from hydroxy, (1-10C)alkoxy, (1-4C)alkoxy-(1-4C)alkoxy, (1-4C)alkoxy-(1-4C)alkoxy-(1-4C)alkoxy, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof] and carboxy. Preferably substituents on a (2-6C)alkyl or (2-4C)alkyl group comprising R₂₁ are 1 or 2 hydroxy groups.

n) Preferably R₂₂ is cyano.

o) Particularly preferred values for AR2, AR2a and AR2b groups are those containing a basic nitrogen, for example pyridine, pyrrolidine, piperazine and piperidine, optionally substituted as hereinbefore defined.

In one embodiment is provided a compound of the formula (Ia) or a pharmaceutically-acceptable salt or an in-vivo hydrolysable ester thereof, 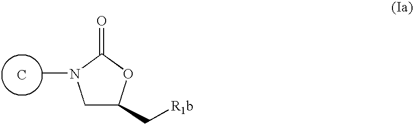
wherein group C is group D; R₂a and R₆a are both hydrogen; R₂b and R₆b are independently hydrogen or fluorine; and R₄ is selected from HET-3.

In another embodiment is provided a compound of the formula (Ia) or a pharmaceutically-acceptable salt or an in-vivo hydrolysable ester thereof, wherein group C is group D; R₂a and R₆a are both hydrogen; R₂b and R₆b are independently hydrogen or fluorine; and R₄ is selected from HET3-T, HET3-U, HET3-V, HET3-W, HET3-X and HET3-Y.

In another embodiment is provided a compound of the formula (Ia) or a pharmaceutically-acceptable salt or an in-vivo hydrolysable ester thereof, wherein group C is group D; R₂a and R₆a are both hydrogen; R₂b and R₆b are independently hydrogen or fluorine; and R₄ is selected from HET3-Z, HET3-AA, HET3-AB, HET3-AC, HET3-AD, HET3-AE, HET3-AF, HET3-AG and HET3-AH.

In another embodiment is provided a compound of the formula (Ia) or a pharmaceutically-acceptable salt or an in-vivo hydrolysable ester thereof, wherein group C is group D; R₂a and R₆a are both hydrogen; R₂b and R₆b are independently hydrogen or fluorine; R₄ is selected from HET3-Z, HET3-AA, HET3-AB, HET3-AC, HET3-AD, HET3-AE, HET3-AF, HET3-AG and HET3-AH; m=1 and R₁a is selected from R₁a3.

In another embodiment is provided a compound of the formula (Ia) or a pharmaceutically-acceptable salt or an in-vivo hydrolysable ester thereof, wherein group C is group E; R₂a and R₆a are both hydrogen; R₂b and R₆b are independently hydrogen or fluorine; and R₄ is selected from HET3-T, HET3-U, HET3-V, HET3-W, HET3-X and HET3-Y.

In another embodiment is provided a compound of the formula (Ia) or a pharmaceutically-acceptable salt or an in-vivo hydrolysable ester thereof, wherein group C is group E; R₂a and R₆a are both hydrogen; R₂b and R₆b are independently hydrogen or fluorine; and R₄ is selected from HET3-Z, HET3-AA, HET3-AB, HET3-AC, HET3-AD, HET3-AE, HET3-AF, HET3-AG and HET3-AH.

In another embodiment is provided a compound of the formula (Ia) or a pharmaceutically-acceptable salt or an in-vivo hydrolysable ester thereof, wherein group C is group E; R₂a and R₆a are both hydrogen; R₂b and R₆b are independently hydrogen or fluorine; R₄ is selected from HET3-Z, HET3-AA, HET3-AB, HET3-AC, HET3-AD, HET3-AE, HET3-AF, HET3-AG and HET3-AH; m=1 and R₁a is is selected from R₁a3

In another embodiment is provided a compound of the formula (Ia) or a pharmaceutically-acceptable salt or an in-vivo hydrolysable ester thereof, wherein group C is group D; R₂a and R₆a are both hydrogen; R₂b and R₆b are independently hydrogen or fluorine; R₄ is selected from HET3-T, HET3-U, HET3-V, HET3-W, HET3-X and HET3-Y, Rz is hydrogen and Z is HET-1A.

In another embodiment is provided a compound of the formula (Ia) or a pharmaceutically-acceptable salt or an in-vivo hydrolysable ester thereof, wherein group C is group D; R₂a and R₆a are both hydrogen; R₂b and R₆b are independently hydrogen or fluorine; and R₄ is selected from HET3-Z, HET3-AA, HET6-AB, HET3-AC, HET3-AD, HET3-AE, HET3-AF, HET3-AG and HET3-AH, Rz is hydrogen and Z is BET-1A.

In another embodiment is provided a compound of the formula (Ia) or a pharmaceutically-acceptable salt or an in-vivo hydrolysable ester thereof, wherein group C is group D; R₂a and R₆a are both hydrogen; R₂b and R₆b are independently hydrogen or fluorine; R₄ is selected from HET3-Z, HET3-AA, HET3-AB, HET3-AC, HET3-AD, HET3-AE, HET3-AF, HET3-AG and HET3-AH; m=1, R₁a is selected from R₁a3, Rz is hydrogen and Z is HET-1A.

In another embodiment is provided a compound of the formula (Ia) or a pharmaceutically-acceptable salt or an in-vivo hydrolysable ester thereof, wherein group C is group E; R₂a and R₆a are both hydrogen; R₂b and R₆b are independently hydrogen or fluorine; and R₄ is selected from HET3-T, HET3-U, HET3-V, HET3-W, HET3-X and HET3-Y, Rz is hydrogen and Z is HET-1A.

In another embodiment is provided a compound of the formula (Ia) or a pharmaceutically-acceptable salt or an in-vivo hydrolysable ester thereof, wherein group C is group E; R₂a and R₆a are both hydrogen; R₂b and R₆b are independently hydrogen or fluorine; and R₄ is selected from HET3-Z, HET3-AA, HET3-AB, HET3-AC, HET3-AD, HET3-AE, HET3-AF, HET3-AG and HET3-AH, Rz is hydrogen and Z is HET-1A.

In another embodiment is provided a compound of the formula (Ia) or a pharmaceutically-acceptable salt or an in-vivo hydrolysable ester thereof, wherein group C is group B; R₂a and R₆a are both hydrogen; R₂b and R₆b are independently hydrogen or fluorine; R₄ is selected from HET3-Z, HET3-AA, HET3-AB, HET3-AC, HET3-AD, HET3-AE, HET3-AF, HET3-AG and HET3-AH; m=1, R₁a is selected from R₁a3, Rz is hydrogen and Z is HET-1A.

In another embodiment is provided a compound of the formula (Ia) or a pharmaceutically-acceptable salt or an in-vivo hydrolysable ester thereof, wherein group C is group E; R₂a and R₆a are both hydrogen; R₂b and R₆b are independently hydrogen or fluorine; R₄ is HET3-V, Rz is hydrogen or —COO(1-4C)alkyl; and Z is selected from isoxazolyl, 1,2,5-thiadiazolyl and isothiazolyl.

In another embodiment is provided a compound of the formula (Ia) or a pharmaceutically-acceptable salt or an in-vivo hydrolysable ester thereof, wherein group C is group E; R₂a and R₆a are both hydrogen; R₂b and R₆b are independently hydrogen or fluorine; R₄ is HET3-V, Rz is hydrogen, and Z is selected from isoxazolyl, 1,2,5-thiadiazolyl and isothiazolyl.

In all of the above definitions the preferred compounds are as shown in formula (Ia).

Particular compounds of the present invention include each individual compound described in the Examples, each of which provides an independent aspect of the invention.

Process Section:

In a further aspect the present invention provides a process for preparing a compound of invention or a pharmaceutically-acceptable salt or an in-vivo hydrolysable ester thereof. It will be appreciated that during certain of the following processes certain substituents may require protection to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon. Resins may also be used as a protecting group.

The protecting groups may be removed at any convenient stage in the synthesis using conventional techniques well known in the chemical art.

A compound of the invention, or a pharmaceutically-acceptable salt or an in vivo hydrolysable ester thereof, may be prepared by any process known to be applicable to the preparation of chemically-related compounds. Such processes, when used to prepare a compound of the invention, or a pharmaceutically-acceptable salt or an in vivo hydrolysable ester thereof, are provided as a further feature of the invention and are illustrated by the following representative examples. Necessary starting materials may be obtained by standard procedures of organic chemistry (see, for example, Advanced Organic Chemistry (Wiley-Interscience), Jerry March or Houben-Weyl, Methoden der Organischen Chemie). The preparation of such starting materials is described within the accompanying non-limiting Examples. Alternatively, necessary starting materials are obtainable by analogous procedures to those illustrated which are within the ordinary skill of an organic chemist. Information on the preparation of necessary starting materials or related compounds (which may be adapted to form necessary starting materials) may also be found in the certain Patent Application Publications, the contents of the relevant process sections of which are hereby incorporated herein by reference; for example WO 94-13649; WO 98-54161; WO 99-64416; WO 99-64417; WO 00-21960; WO 01-40222.

In particular we refer to our PCT patent applications WO 99/64417 and WO 00/21960 wherein detailed guidance is given on convenient methods for preparing oxazolidinone compounds.

The skilled organic chemist will be able to use and adapt the information contained and referenced within the above references, and accompanying Examples therein and also the Examples herein, to obtain necessary starting materials, and products. For example, the skilled chemist will be able to apply the teaching herein for compounds of formula (I) in which a pyrimidyl-phenyl group is present (that is when group C is group D) to prepare compounds in which a pyridyl-phenyl group is present (that is when group C is group E) as heereinbefore defined and vice versa.

Thus, the present invention also provides that the compounds of the invention and pharmaceutically-acceptable salts and in-vivo hydrolysable esters thereof, can be prepared by a process (a) to (f); and thereafter if necessary:

i) removing any protecting groups;

ii) forming a pro-drug (for example an in-vivo hydrolysable ester); and/or

iii) forming a pharmaceutically-acceptable salt;

wherein said processes (a) to (f) are as follows (wherein the variables are as defined above unless otherwise stated):

(wherein the variables are as defined above unless otherwise stated):

a) by modifying a substituent in, or introducing a substituent into another compound of the invention by using standard chemistry (see for example, Comprehensive Organic Functional Group Transformations (Pergamon), Katritzky, Meth-Cohn & Rees or Advanced Organic Chemistry (Wiley-Interscience), Jerry March or Houben-Weyl, Methoden der Organischen Chemie); for example:

an acylamino group may be converted into a thioacylamino group either directly or through the intermediacy of the corresponding amino group;

an acylamino group or thioacylamino group may be converted into another acylamino or thioacylamino; heterocyclyl for instance tetrazolyl or thiazolyl, or heterocyclylamino group (optionally substituted or protected on the amino-nitrogen atom) either directly or through the intermediacy of one or more derivatives such as the corresponding amino group;

an acyloxy group may be converted into a hydroxy group or into the groups that may be obtained from a hydroxy group (either directly or through the intermediacy of a hydroxy group);

an alkyl halide such as alkylbromide or alkyliodide may be converted into an alkyl fluoride or nitrile;

an alkyl sulfonate such as alkyl methanesulfonate may be converted into an alkyl fluoride or nitrile;

an alkylthio group such as methylthio may be converted into a methanesulfinyl or methanesulfonyl group;

an arylthio group such as phenylthio may be converted into a benzenesulfinyl or benzenesulfonyl group;

an amidino or guanidino group may be converted into a range of 2-substituted 1,3-diazoles and 1,3-diazines;

an amino group may be converted for instance into acylamino or thioacylamino for instance an acetamide (optionally substituted), alkyl- or dialkyl-amino and thence into a further range of N-alkyl-amine derivatives, sulfonylamino, sulfinylamino, amidino, guanidino, arylamino, heteroarylamino;

an aryl- or heteroary-halide group such as an aryl- or hetero-aryl chloride or bromide or iodide may be converted by transition metal mediated coupling, especially Pd(0) mediated coupling into a range of aryl-, heteroaryl, alkenyl, alkynyl, acyl, alkylthio, or alkyl- or dialkyl-amino substituted aryl or heteroaryl groups;

an aryl- or heteroary-sulfonate group such as an aryl- or hetero-aryl trifluoromethanesulfonate may be converted by transition metal mediated coupling, especially Pd(0) mediated coupling into a range of aryl-, heteroaryl, alkenyl, alkynyl, acyl, alkylthio, or alkyl- or dialkyl-amino substituted aryl or heteroaryl groups;

an aryl- or heteroary-halide group such as an aryl- or hetero-aryl chloride or bromide or iodide may be converted by transition metal mediated coupling, especially Pd(0) mediated coupling into a range of trialkyltin, dialkylboronate, trialkoxysilyl, substituted aryl or heteroaryl groups useful as intermediates for the synthesis of compounds of the invention;

an azido group may be converted for instance into a 1,2,3-triazolyl or amine and thence by methods that are well known in the art into any of the range common amine derivatives such as acylamino for instance acetamido group;

a carboxylic acid group may be converted into trifloromethyl, hydroxymethyl, alkoxycarbonyl, aminocarbonyl optionally substituted on nitrogen, formyl, or acyl groups;

a cyano group may be converted into a tetrazole, or an imidate, an amidine, an amidrazone, an N-hydroxyamidrazone, an amide, a thioamide, an ester, or an acid and thence by methods that are well known in the art into any of the range of heterocycles derived from such nitrile derivatives;

a hydroxy group may be converted for instance into an alkoxy, cyano, azido, alkylthio, keto and oximino, fluoro, bromo, chloro, iodo, alkyl- or aryl-sulfonyloxy for instance trifluoromethanesulfonate, methanesulfonate, or tosylsulfonate, silyloxy; acylamino or thioacylamino, for instance an acetamide (optionally substituted or protected on the amido-nitrogen atom); acyloxy, for instance an acetoxy; phosphono-oxy, heterocyclylamino (optionally substituted or protected on the amino-nitrogen atom), for instance an isoxazol-3-ylamino or a 1,2,5-thiadiazol-3-ylamino; group; such conversions of the hydroxy group taking place directly (for instance by acylation or Mitsunobu reaction) or through the intermediacy of one or more derivatives (for instance a mesylate or an azide);

a silyloxy group may be converted into a hydroxy group or into the groups that may be obtained from a hydroxy group (either directly or through the intermediacy of a hydroxy group);

a keto group may be converted into a hydroxy, thiocarbonyl, oximino, or difluoro group;

a nitro-group may be converted into an amino group and thence by methods that are well known in the art into any of the range common amine derivatives such as acylamino for instance acetamido group;

a 2-, 4, or 6-pyridyl or 2-, 4, or 6-pyrimidyl halide such as chloride or sulfonate such as mesylate substituent may be converted into alkoxy, alkythio, amino, alkylamino, dialkylamino, or N-linked heterocyclic substituents;

moreover, an optionally substituted heteroaromatic ring D or E may be converted into another heteroaromatic ring D or E by introduction of a new substituent (R₂a, R₃a, or R₆a) or by refunctionalisation of an existing substituent (R₂a, R₃a, or R₆a);

a heterocyclyl group linked through nitrogen may be converted into another heterocyclyl group linked through nitrogen by introduction of a new ring substituent or by refunctionalisation of an existing ring substituent;

a heterocyclylamino group may be converted into another heterocyclylamino group by introduction of a new ring substituent or by refunctionalisation of an existing ring substituent;

for instance, examples drawn from the methods for conversion of an hydroxy group into an heteroarylamino group are illustrated by the scheme: 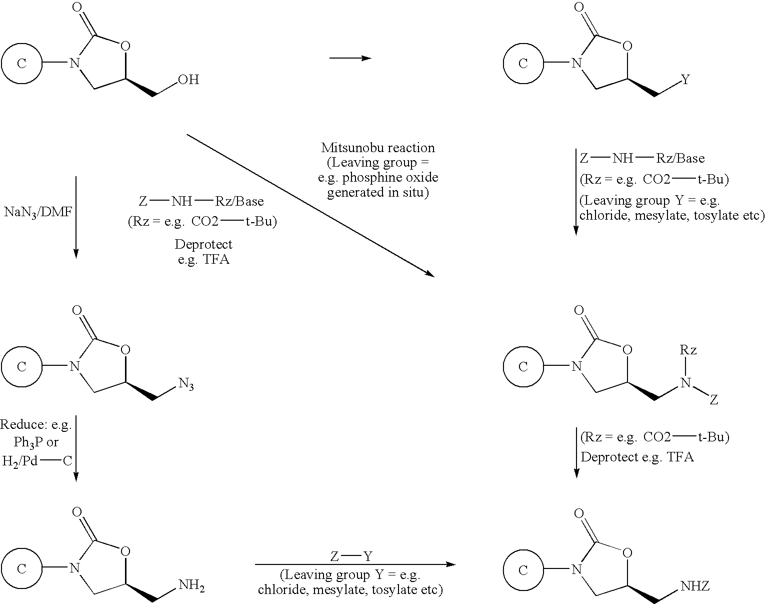
b) by reaction of a molecules of a compound of formula (IIa) [wherein X is a leaving group useful in palladium coupling (for example chloride, bromide, iodide, trifluoromethylsulfonyloxy, trimethylstannyl, trialkoxysilyl, or a boronic acid residue) and in this instance A is either N or C—R₃a] with a molecule of a compound of formula (IIb) (wherein X′ is a leaving group useful in palladium coupling, for example chloride, bromide, iodide, trifluoromethylsulfonyloxy, trimethylstannyl, trialkoxysilyl, or a boronic acid residue) wherein X and X′ are chosen such that an aryl-aryl, heteroaryl-aryl, or heteroaryl-heteroaryl bond replaces the aryl-X (or heteroaryl-X) and aryl-X′ (or heteroaryl-X′) bonds; such methods are now well known, see for instance J. K. Stille, Angew Chem. Int. Ed. Eng., 1986, 25, 509-524; N. Miyaura and A Suzuki, Chem. Rev., 1995, 95, 2457-2483, D. Baranano, G. Mann, and J. F. Hartwig, Current Org. Chem., 1997, 1, 287-305, S. P. Stanforth, Tetrahedron, 54 1998, 263-303, and P. R. Parry, C. Wang, A. S. Batsanov, M. R. Bryce, and B. Tarbit, J. Org. Chem., 2002, 67, 7541-7543; 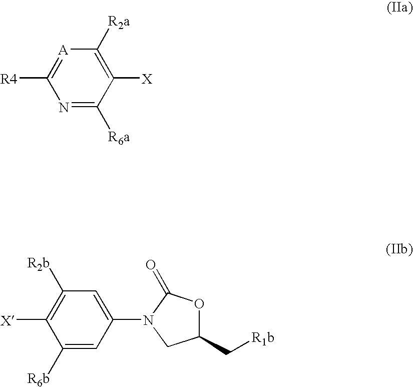
the leaving groups X and X′ are chosen to be different and to lead to the desired cross-coupling products of formula (I);
for example 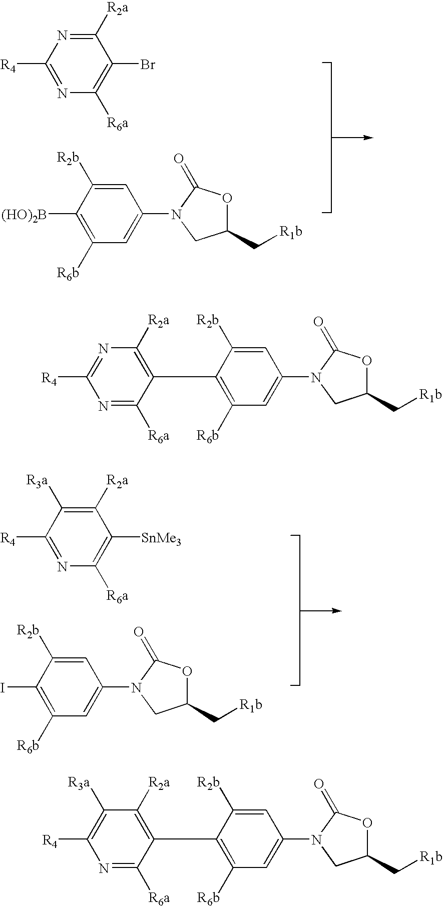
the pyridines, pyrimidines, and aryl oxazolidinones required as reagents for process b) or as intermediates for the preparation of reagents for process b) may be prepared by standard organic methods, for instance by methods analogous to those set out in process sections c) to j); methods for the introduction and interconversion of Groups X and X′ are well known in the art;
c) by reaction of a heterobiaryl derivative (III) carbamate [where in this instance A is either N or C—R₃a] with an appropriately substituted oxirane to form an oxazolidinone ring; 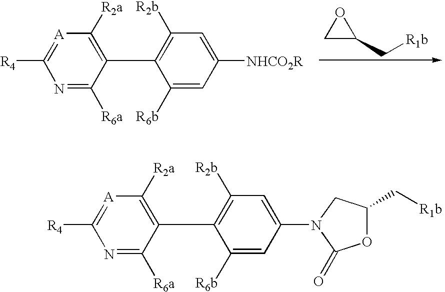
variations on this process in which the carbamate is replaced by an isocyanate or by an amine or/and in which the oxirane is replaced by an equivalent reagent X—CH₂CH(O-optionally protected)CH₂R₁b where X is a displaceable group are also well known in the art, for example, 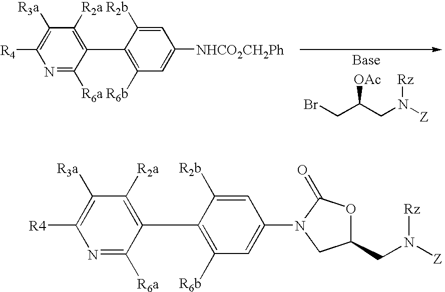
(d) by reaction of a compound of formula (VI): 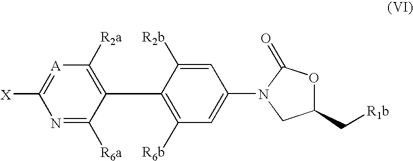
where X is a replaceable substituent—such as chloride, bromide, iodide, trifluoromethylsulfonyloxy, trimethylstannyl, trialkoxysilyl, or a boronic acid residue and A is either N or CR₃a with a compound of the formula (VII):
T-X′ (VII)
wherein T-X′ is HET3 as herein above defined and X′ is a replaceable C-linked substituent—such as chloride, bromide, iodide, trifluoromethylsulfonyloxy, trimethylstannyl, trialkoxysilyl, or a boronic acid residue; wherein the substituents X and X′ are chosen to be complementary pairs of substituents known in the art to be suitable as complementary substrates for coupling reactions catalysed by transition metals such as palladium(0); or
(d(i)) by reaction catalysed by transition metals such as palladium(0) of a compound of formula (VIII): 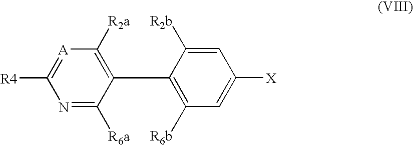
wherein X is a replaceable substituent—such as chloride, bromide, iodide, trifluoromethylsulfonyloxy, trimethylstannyl, trialkoxysilyl, or a boronic acid residue and wherein in this instance A is either N or C—R₃a with a compound of the formula (IX) (Tetrahedron Letts., 2001, 42(22), 3681-3684): 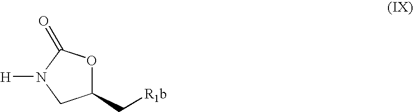
(d(ii)) by reaction of a compound of formula (X): 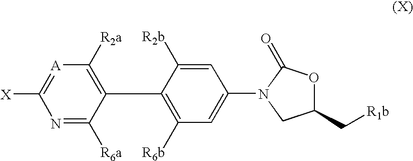
wherein X is a replaceable substituent—such as chloride, bromide, iodide, trifluoromethylsulfonyloxy—and wherein in this instance A is either N or C—R₃a with a compound of the formula (XI):
T-H (XI)
wherein T-H is an amine R₇R₈NH, an alcohol R₁₀OH, or an azole with an available ring-NH group to give compounds (XIIa), (XIIb), or (XIIc) wherein in this instance A is nitrogen or C—R₃a and A′ is nitrogen or carbon optionally substituted with one or more groups R₁a; 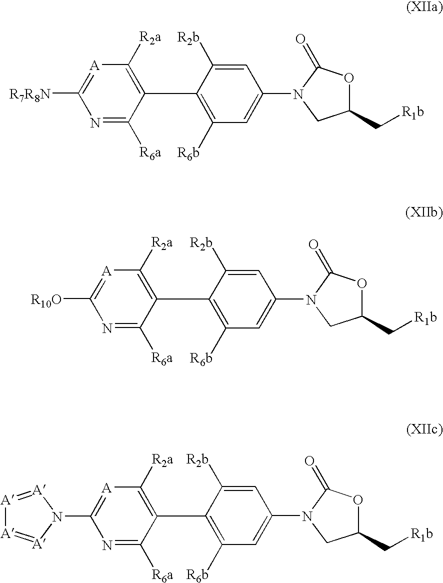
(e) by reaction of a compound of formula (XIII): 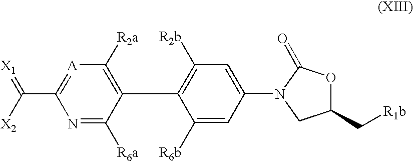
wherein X₁ and X₂ here are independently optionally substituted heteroatoms drawn in combination from O, N, and S such that C(X₁)X₂ constitutes a substituent that is a carboxylic acid derivative substituent and wherein in this instance A is either N or C—R₃a with a compound of the formula (XIV) and X₃ and X₄ are independently optionally substituted heteroatoms drawn in combination from O, N, and S: 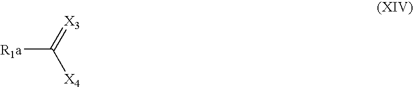
and wherein one of C(X₁)X₂ and C(X₃)X₄ constitutes an optionally substituted hydrazide, thiohydrazide, or amidrazone, hydroximidate, or hydroxamidine and the other one of C(X₁)X₂ and C(X₃)X₄ constitutes an optionally substituted acylating, thioacylating, or imidoylating agent such that C(X₁)X₂ and C(X₃)X₄ may be condensed together to form a 1,2,4-heteroatom 5-membered heterocycle containing 3 heteroatoms drawn in combination from O, N, and S, for instance thiadiazole, by methods well-known in the art; or
(e(i)) by reaction of a compound of formula (XV): 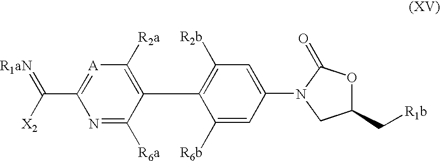
wherein X₂ is a displaceable group such as ethoxy or diphenylphosphonyloxy and wherein and wherein in this instance A is either N or C—R₃a with a source of azide anion such as sodium azide to give a tetrazole (XVI); 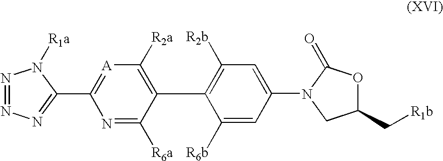
alternatively nitriles of formula (XVII) 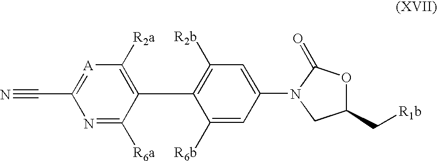
[wherein in this instance A is either N or C—R₃a] may be reacted directly with azides such as ammonium azide or trialkylstannylazides to give tetrazoles (XVI, R1a=H) that are subsequently alkylated with groups R1a≠H to give tetrazoles (XVIIIa) and (XVIIIb) or; 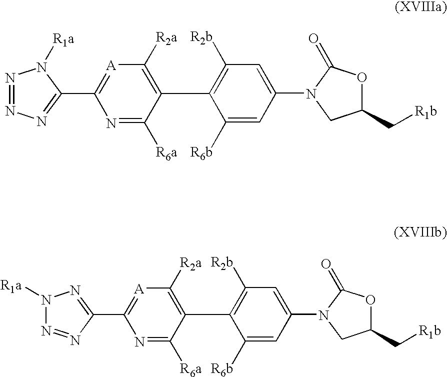
(f) by reaction of a compound of formula (XIX): 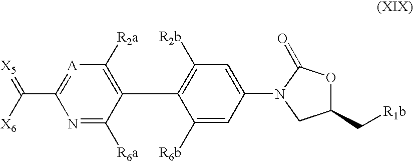
[wherein in this instance A is either N or C—R₃a] with a compound of the formula (XX): 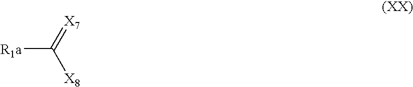
wherein one of C(X₅)X₆ and C(X₇)X₈ constitutes an optionally substituted alpha-(leaving-group-substituted)ketone, wherein the leaving group is for example a halo-group or an (alkyl or aryl)-sulfonyloxy-group, and the other one of C(X₅)X₆ and C(X₇)X₈ constitutes an optionally substituted amide, thioamide, or amidine, such that C(X₅)X₆ and C(X₇)X₈ are groups that may be condensed together to form a 1,3-heteroatom 5-membered heterocycle containing 2 heteroatoms drawn in combination from O, N, and S, for instance thiazole, by methods well-known in the art.

The removal of any protecting groups, the formation of a pharmaceutically-acceptable salt and/or the formation of an in-vivo hydrolysable ester are within the skill of an ordinary organic chemist using standard techniques. Furthermore, details on the these steps, for example the preparation of in-vivo hydrolysable ester prodrugs has been provided, for example, in the section above on such esters.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

According to a further feature of the invention there is provided a compound of the invention, or a pharmaceutically-acceptable salt, or in-vivo hydrolysable ester thereof for use in a method of treatment of the human or animal body by therapy.

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt, or in-vivo hydrolysable ester thereof.

The invention also provides a compound of the invention, or a pharmaceutically-acceptable salt, or in-vivo hydrolysable ester thereof, for use as a medicament; and the use of a compound of the invention of the present invention, or a pharmaceutically-acceptable salt, or in-vivo hydrolysable ester thereof, in the manufacture of a medicament for use in the production of an antibacterial effect in a warm blooded animal, such as man.

In order to use a compound of the invention, an in-vivo hydrolysable ester or a pharmaceutically-acceptable salt thereof, including a pharmaceutically-acceptable salt of an in-vivo hydrolysable ester, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the invention, an in-vivo hydrolysable ester or a pharmaceutically-acceptable salt thereof, including a pharmaceutically-acceptable salt of an in-vivo hydrolysable ester, and a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration as eye-drops, for administration by inhalation (for example as a finely divided powder or a liquid aerosol), for administration by insufflation (for example as a finely divided powder) or for parenteral administration (for example as a sterile aqueous or oily solution for intravenous, subcutaneous, sub-lingual, intramuscular or intramuscular dosing or as a suppository for rectal dosing).

In addition to the compounds of the present invention, the pharmaceutical composition of this invention may also contain (ie through co-formulation) or be co-administered (simultaneously, sequentially or separately) with one or more known drugs selected from other clinically useful antibacterial agents (for example, β-lactams, macrolides, quinolones or aminoglycosides) and/or other anti-infective agents (for example, an antifungal triazole or amphotericin). These may include carbapenems, for example meropenem or imipenem, to broaden the therapeutic effectiveness. Compounds of this invention may also be co-formulated or co-administered with bactericidal/permeability-increasing protein (BPI) products or efflux pump inhibitors to improve activity against gram negative bacteria and bacteria resistant to antimicrobial agents. Compounds of this invention may also be co-formulated or co-administered with a vitamin, for example Vitamin B, such as Vitamin B2, Vitamin B6, Vitamin B12 and folic acid. Compounds of the invention may also be formulated or co-administered with cyclooxygenase (COX) inhibitors, particularly COX-2 inhibitors.

In one aspect of the invention, a compound of the invention is co-formulated with an antibacterial agent which is active against gram-positive bacteria.

In another aspect of the invention, a compound of the invention is co-formulated with an antibacterial agent which is active against gram-negative bacteria.

In another aspect of the invention, a compound of the invention is co-administered with an antibacterial agent which is active against gram-positive bacteria.

In another aspect of the invention, a compound of the invention is co-administered with an antibacterial agent which is active against gram-negative bacteria.

The compositions of the invention may be obtained by conventional procedures using conventional pharmaceutical excipients, well known in the art. Thus, compositions intended for oral use may contain, for example, one or more colouring, sweetening, flavouring and/or preservative agents. A pharmaceutical composition to be dosed intravenously may contain advantageously (for example to enhance stability) a suitable bactericide, antioxidant or reducing agent, or a suitable sequestering agent.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol. Solubility enhancing agents, for example cyclodextrins may be used.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 50 mg to 5 g of active agent compounded with an appropriate and convenient amount of excipients which may vary from about 5 to about 98 percent by weight of the total composition. Dosage unit forms will generally contain about 200 mg to about 2 g of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

A suitable pharmaceutical composition of this invention is one suitable for oral administration in unit dosage form, for example a tablet or capsule which contains between 1 mg and 1 g of a compound of this invention, preferably between 100 mg and 1 g of a compound. Especially preferred is a tablet or capsule which contains between 50 mg and 800 mg of a compound of this invention, particularly in the range 100 mg to 500 mg.

In another aspect a pharmaceutical composition of the invention is one suitable for intravenous, subcutaneous or intramuscular injection, for example an injection which contains between 0.1% w/v and 50% w/v (between 1 mg/ml and 500 mg/ml) of a compound of this invention.

Each patient may receive, for example, a daily intravenous, subcutaneous or intramuscular dose of 0.5 mgkg⁻¹ to 20 mgkg⁻¹ of a compound of this invention, the composition being administered 1 to 4 times per day. In another embodiment a daily dose of 5 mgkg⁻¹ to 20 mgkg⁻¹ of a compound of this invention is administered. The intravenous, subcutaneous and intramuscular dose may be given by means of a bolus injection. Alternatively the intravenous dose may be given by continuous infusion over a period of time. Alternatively each patient may receive a daily oral dose which may be approximately equivalent to the daily parenteral dose, the composition being administered 1 to 4 times per day.

In the above other, pharmaceutical composition, process, method, use and medicament manufacture features, the alternative and preferred embodiments of the compounds of the invention described herein also apply.

Antibacterial Activity:

The pharmaceutically-acceptable compounds of the present invention are useful antibacterial agents having a good spectrum of activity in vitro against standard Gram-positive organisms, which are used to screen for activity against pathogenic bacteria Notably, the pharmaceutically-acceptable compounds of the present invention show activity against enterococci, pneumococci and methicillin resistant strains of S.aureus and coagulase negative staphylococci, together with haemophilus and moraxella strains. The antibacterial spectrum and potency of a particular compound may be determined in a standard test system.

The (antibacterial) properties of the compounds of the invention may also be demonstrated and assessed in-vivo in conventional tests, for example by oral and/or intravenous dosing of a compound to a warm-blooded mammal using standard techniques.

The following results were obtained on a standard in-vitro test system. The activity is described in terms of the minimum inhibitory concentration (MIC) determined by the agar-dilution technique with an inoculum size of 10⁴ CFU/spot. Typically, compounds are active in the range 0.01 to 256 μg/ml.

Staphylococci were tested on agar, using an inoculum of 10⁴ CFU/spot and an incubation temperature of 37° C. for 24 hours—standard test conditions for the expression of methicillin resistance.

Streptococci and enterococci were tested on agar supplemented with 5% defibrinated horse blood, an inoculum of 10⁴ CFU/spot and an incubation temperature of 37° C. in an atmosphere of 5% carbon dioxide for 48 hours—blood is required for the growth of some of the test organisms. Fastidious Gram negative organisms were tested in Mueller-Hinton broth, supplemented with hemin and NAD, grown aerobically for 24 hours at 37° C., and with an innoculum of 5×10⁴ CFU/well.

For example, the following results were obtained for the compound of Example 2:

| Organism | | MIC (μg/ml) |
|---|---|---|
| Staphylococcus aureus: | MSQS | 0.5 |
| | MRQR | 0.5 |
| Streptococcus pneumoniae | | 0.13 |
| Enterococcus faecium | | 0.5 |
| Haemophilus influenzae | | 64 |
| Moraxella catarrhalis | | 0.5 |
| Linezolid Resistant Streptococcus pneumoniae | | 2 |

MSQS = methicillin sensitive and quinolone sensitive
MRQR = methicillin resistant and quinolone resistant

Certain intermediates and/or Reference Examples described hereinafter are within the scope of the invention and may also possess useful activity, and are provided as a further feature of the invention.

The invention is now illustrated but not limited by the following Examples in which unless otherwise stated:—

(i) evaporations were carried out by rotary evaporation in-vacuo and work-up procedures were carried out after removal of residual solids by filtration;

(ii) operations were carried out at ambient temperature, that is typically in the range 18-26° C. and without exclusion of air unless otherwise stated, or unless the skilled person would otherwise work under an inert atmosphere;

(iii) column chromatography (by the flash procedure) was used to purify compounds and was performed on Merck Kieselgel silica (Art. 9385) unless otherwise stated;

(iv) yields are given for illustration only and are not necessarily the maximum attainable;

(v) the structure of the end-products of the invention were generally confirmed by NMR and mass spectral techniques [proton magnetic resonance spectra were generally determined in DMSO-d₆ unless otherwise stated using a Varian Gemini 2000 spectrometer operating at a field strength of 300 MHz, or a Bruker AM250 spectrometer operating at a field strength of 250 MHz; chemical shifts are reported in parts per million downfield from tetramethysilane as an internal standard (δ scale) and peak multiplicities are shown thus: s, singlet; d, doublet; AB or dd, doublet of doublets; dt, doublet of triplets; dm, doublet of multiplets; t, triplet, m, multiplet; br, broad; fast-atom bombardment NAB) mass spectral data were generally obtained using a Platform spectrometer (supplied by Micromass) run in electrospray and, where appropriate, either positive ion data or negative ion data were collected]; optical rotations were determined at 589 nm at 20° C. for 0.1M solutions in methanol using a Perkin Elmer Polarimeter 341;

(vi) each intermediate was purified to the standard required for the subsequent stage and was characterised in sufficient detail to confirm that the assigned structure was correct; purity was assessed by HPLC, TLC, or NMR and identity was determined by infra-red spectroscopy (IR), mass spectroscopy or NMR spectroscopy as appropriate;

(vii) in which the following abbreviations may be used:—

DMF is N,N-dimethylformamide; DMA is N,N-dimethylacetamide; TLC is thin layer chromatography; HPLC is high pressure liquid chromatography; MPLC is medium pressure liquid chromatography; NMP is N-methylpyrrolidone; DMSO is dimethylsulfoxide; CDCl₃ is deuterated chloroform; MS is mass spectroscopy; ESP is electrospray; EI is electron impact; CI is chemical ionisation; APCI is atmospheric pressure chemical ionisation; EtOAc is ethyl acetate; MeOH is methanol; phosphoryl is (HO)₂—P(O)—O—; phosphiryl is (HO)₂—P—O—; Bleach is “Clorox” 6.15% sodium hypochlorite;

(viii) temperatures are quoted as ° C.

### EXAMPLE 1

### O-tert-Butyl N-(5R)-3-{3-fluoro-4-[6-(2-methyl-2H-tetrazol-5-yl)pyridin-3-yl]phenyl}-1,3-oxazolidin-2-on-5-yl)methyl N-isoxazol-3-ylcarbamate

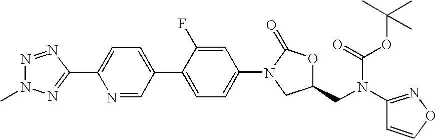 A stirred solution of O-tert-butyl N-(5R)-3-{3-fluoro-4-iodophenyl}-1,3-oxazolidin-2-on-5-yl)methyl N-isoxazol-3-ylcarbamate (637 mg, 1.15 mmol) and copper (I) iodide (99 mg, 0.5 mmol) in dry 1-methyl-2-pyrrolidinone (2 mL) was degassed and maintained under an atmosphere of argon. The solution was treated with tetrakis(triphenylphosphine)palladium(0) (146 mg, 0.13 mmol) and then with a solution of 2-(2-methyl-2H-tetrazol-5-yl)-5-(trimethylstannyl)pyridine (410 mg, 1.26 mmol) in 1-methyl-2-pyrrolidinone (2 mL). The reaction mixture stirred for 16 hours at 90° C. and then treated with water (20 mL) and ethyl acetate (20 mL). The mixture was filtered and the phases were separated. The ethyl acetate phase dried over magnesium sulphate, concentrated in vacuo onto silica gel (2 g), applied to silica-gel column (SiO₂ 50 g bond elut) and then eluted (gradient of 40% to 100% ethyl acetate:hexanes) to give the title compound (483 mg, 71%).

MS (ESP+): (M+H)⁺ 537.38 for C₂₅H₂₅FN₈O₅

NMR (DMSO-d₆) δ: 1.52 (s, 9H); 3.94 to 4.07 (m, 2H); 4.29 to 4.37 (m, 2H); 4.50 (s, 3H); 5.08 (m, 1H); 6.90 (s, 1H); 7.55 (dd, 1H); 7.70 (dd, 1H); 7.80 (t, 1H); 8.25 (m, 2H); 8.85 (d, 1H); 8.97 (s, 1H).

The intermediates for this compound were prepared as follows:—

### 3-Bromo-6-cyano-pyridine

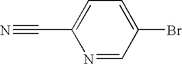 A stirred solution of 2,5-dibromopyridine (39.465 g, 0.17 mol) in anhydrous NMP (100 mL) was treated with CuCN (14.42 g, 0.17 mol) for 20 hours at 110° C. under nitrogen. The reaction mixture was cooled to 40° C. and treated with aqueous sodium hydroxide (2M; 200 mL) and then with ethyl acetate (200 mL). The mixture was stirred for 1 hour and then filtered through Celite to remove the resulting precipitate. The retained solid was washed with aqueous sodium hydroxide (2M; 600 mL) and then with ethyl acetate (600 mL). The organic layers were combined and washed with aqueous ammonium hydroxide (5M; 800 mL), dried over magnesium sulfate, and evaporated to dryness under reduced pressure. The involatile residue was purified by chromatography on silica gel [elution gradient 1% to 7% of ethyl acetate in hexanes] to give the title compound (8.538 g, 28%), as a colorless amorphous solid.

¹H-NMR (DMSO-d₆) (300 MHz) δ 8.05 (d, 1H); 8.40 (dd, 1H); 8.95 (d, 1H).

### 5-Bromo-2-tetrazol-5-ylpyridine

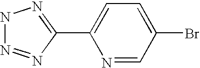 A mixture of 3-bromo-6-cyano-pyridine (2 g, 10.9 mmol), sodium azide (0.85 g, 13 mmol), and ammonium chloride (0.59 g, 11 mmol) in N,N-dimethylformamide (20 mL) was heated for 1 h at 120° C. The reaction mixture was diluted with ethyl acetate (˜100 mL) and the product was isolated by filtration and then washed with ethyl acetate to give the title compound, an off-white amorphous solid which was used in the next step without further purification.

### 5-Bromo-2-(2-methyl-2H-tetrazol-5-yl)pyridine and 5-bromo-2-(1-methyl-1H-tetrazol-5-yl)pyridine

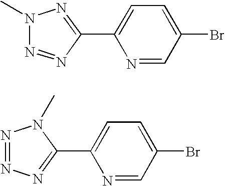 5-Bromo-2-(2-methyl-2H-tetrazol-5-yl)pyridine and 5-bromo-2-(1-methyl-1H-tetrazol-5-yl)pyridine were prepared according to the procedure described by Dong A Pharmaceuticals (WO 01/94342).
A mixture of 6.5 g unpurified 5-bromo-2-tetrazol-5-ylpyridine [Dong A Pharmaceuticals (WO 01/94342)] (˜28 mmol) and sodium hydroxide (9 g, 125 mmol) in dry DMF was evaporated to dryness under reduced pressure. A stirred solution of the involatile residue in dry DMP (50 mL)was treated dropwise at ice-bath temperature with iodomethane (3.0 mL, 48 mmol). The stirred reaction mixture was allowed to warm and then maintained at room temperature for 2 hours. The reaction mixture was partitioned between iced water and ethyl acetate. The organic phase was washed with water, dried over magnesium sulfate, and tehn evaporated under reduced pressure to give a residue that was purified by chromatography on silica gel [elution with dichloromethane:ethyl acetate (60:1)] to give:
1. 5-bromo-2-(1-methyl-1H-tetrazol-5-yl)pyridine (1.397 g), a colorless solid, (TLC: silica-gel, hexanes:ethyl acetate (4:1), Rf: 0.3), ¹H-NMR-(DMSO-d₆) (300 MHz) δ: 4.38 (s, 3H); 8.17 (d, 1H); 8.35 (dd, 1H); 8.96 (d, 1H).
2. 5-bromo-2-(2-methyl-2H-tetrazol-5-yl)pyridine (1.07 g), a colorless solid, (TLC: silica-gel, hexanes:ethyl acetate (4:1), Rf: 0.1). ¹H-NMR (DMSO-d₆) (300 MHz) δ 4.46 (s, 3H); 8.09 (d, 1H); 8.28 (dd, 1H); 8.88 (d, 1H).
   Structure assignment based on HMBC experiments, in which long range coupling of the protons of CH₃ to the C5 of the tetrazole ring is observed in the 1-methyl-1H-isomer of Rf 0.3, but not in the 2-methyl-2H-isomer of Rf 0.1). The compound referred to as 5-bromo-2-(1-methyl-1H-tetrazol-5-yl)pyridine is thus the isomer of Rf 0.3 and the compound referred to as 5-bromo-2-(2-methyl-2H-tetrazol-5-yl)pyridine is thus the isomer of Rf 0.1

### 2-(2-Methyl-2H-tetrazol-5-yl)-5-(trimethylstannyl)pyridine

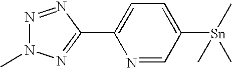 A stirred solution of 5-bromo-2-(2-methyl-1H-tetrazol-5-yl)pyridine (0.919 g, 3.80 mmol) in dry 1,4-dioxane (30 mL) was degassed and maintained under an atmosphere of argon. The solution was treated with hexamethylditin (1.38 g, 4.20 mmol) and then bis(triphenylphosphine)palladium(II) chloride (0.134 g, 0.19 mmol). The reaction mixture was stirred at 90° C. for 18 hours under an atmosphere of argon and then evaporated by dryness. A solution of the involatile residue in ethyl acetate (50 mL) was treated with Isolute HM-N (10 mL) and then evaporated to dryness to give a residue that was purified by column chromatography (SiO₂ 50 g bond elut, elution gradient 0% to 50% ethyl acetate:hexanes) to yield the title compound (0.842 g, 68%) as a white solid.

MS (ESP): (M+H)⁺ 322.39, 324.39, 326.40, 328.40 & 330.40 for C₁₀H₁₅N₅Sn

NMR (DMSO-d₆) δ: 0.37 (s, 9H); 4.47 (s, 3H); 8.06 to 8.16 (m, 2H); 8.78 (m, 1H).

### (5R)-3-(3-Fluoro-4-iodophenyl)-5-hydroxymethyloxazolidin-2-one

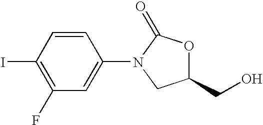 Acetic acid (5R)-3-(3-fluoro-4-iodophenyl)oxazolidin-2-on-5-ylmethyl ester (30 g, 79 mmol) was treated with potassium carbonate (16.4 g, 0.119 mmol) in a mixture of methanol (800 mL) and dichloromethane (240 mL) at ambient temperature for 25 minutes, then immediately neutralised by the addition of acetic acid (10 mL) and water (500 mL). The precipitate was filtered, washed with water, and dissolved in dichloromethane (1.2 L), the solution washed with saturated sodium bicarbonate, and dried (magnesium sulfate). Filtration and evaporation gave the desired product (23 g).

MS (ESP): 338 (MH⁺) for C₁₀H₉FINO₃

NMR (DMSO-d₆) δ: 3.53 (m, 1H); 3.67 (m, 1H); 3.82 (dd, 1H); 4.07 (t, 1H); 4.70 (m, 1H); 5.20 (t, 1H); 7.21 (dd, 1H); 7.57 (dd, 1H); 7.81 (t, 1H).

### Methanesulfonic acid (5R)-3-(3-fluoro-4-iodo-phenyl)-2-oxo-oxazolidin-5-ylmethyl ester

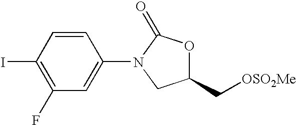 (5R)-3-(3-Fluoro-4-iodophenyl)-5-hydroxymethyl)oxazolidin-2-one (6.07 g, 18 mmol) in dry dichloromethane (200 mL) under nitrogen was treated with triethylamine (2.54 g, 25 mmol), and methanesulfonyl chloride (2.47 g, 22 mmol) run in dropwise over 30 minutes at 0° C. After stirring 2 hours at 0° C., the mixture was diluted with water (200 mL), the organic layer separated, washed with hydrochloric acid (2N, 100 mL), sodium bicarbonate solution (5%, 100 mL), brine (100 mL) and dried (magnesium sulfate). The residue after evaporation was dissolved in the minimum of dichloromethane, and excess isohexane added to precipitate the desired product (7.05 g).

MS (ESP): 416 (MH⁺) for C₁₁H₁₁FINO₅S

NMR (DMSO-d₆) δ: 3.26 (s, 3H); 3.81 (dd, 1H); 4.18 (t, 1H); 4.46 (dd, 1H); 4.53 (dd, 1H); 5.01 (m, 1H); 7.20 (dd, 1H); 7.57 (dd, 1H); 7.83 (t, 1H).

### O-tert-Butyl N-(5R)-3-{3-fluoro-4-iodophenyl}-1,3-oxazolidin-2-on-5-yl)methyl N-isoxazol-3-ylcarbamate

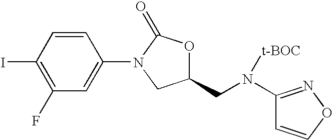 A solution of N-isoxazol-3-yl-carbamic acid tert-butyl ester (1.66 g, 9.04 mmol) in N,N-dimethylformamide (10 mL) was added dropwise at ambient temperature to a stirred suspension of sodium hydride (60% in oil, 9.04 mol) in dry N,N-dimethylformamide (10 mL) under an atmosphere of nitrogen. The mixture was treated slowly with a solution of methane sulfonic acid (5R)-3-(3-fluoro-4-iodo-phenyl)oxazolidin-2-one-5-ylmethyl ester (2.5 g, 6.02 mmol) in N,N-dimethylformamide (10 mL) and the reaction mixture was heated to 75° C. for 2 hours. After cooling, the mixture was diluted with aqueous sodium bicarbonate (5%, 300 mL), and extracted with ethyl acetate (3×100 mL). The organic phase was washed with water (100 mL) and brine (100 mL), dried (magnesium sulfate), evaporated and crude product purified by chromatography on a 50 g silica Mega Bond Elut® column, eluting with dichloromethane. Relevant fractions were combined to give the title compound (1.73 g).

MS (ESP): 504 (MH⁺) for C₁₈H₁₉FIN₃O₅

NMR (DMSO-d₆) δ: 1.47 (s, 9H); 3.84 (dd, 1H); 3.96 (dd, 1H); 4.19 (t, 1H); 4.99 (dd, 1H); 4.99 (m, 1H); 6.83 (d, 1H); 7.19 (dd, 1H); 7.53 (dd, 1H); 7.82 (dd, 1H); 8.79 (d, 1H).

### EXAMPLE 2

### (5S)-3-{3-Fluoro-4-[6-(2-methyl-2H-tetrazol-5-yl)pyridin-3-yl]phenyl}-5-isoxazol-3-ylaminomethyl)-1,3-oxazolidin-2-one

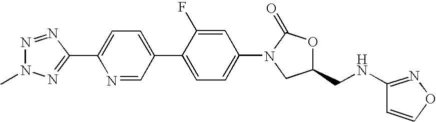 A solution of O-tert-butyl N-(5R)-3-{3-fluoro-4-[6-(2-methyl-2H-tetrazol-5-yl)pyridin-3-yl]phenyl}-1,3-oxazolidin-2-on-5-yl)methyl N-isoxazol-3-ylcarbamate (0.10 g, 0.19 mmol) in a mixture of trifluoroacetic acid (2 mL) and dichloromethane (1 mL) was stirred at room temperature for 30 minutes. The reaction mixture was concentrated in vacuo and the involatile residue was redissolved in DMSO (1 mL) and then purified by reverse phase preparative HPLC [gradient from 30% acetonitrile:water to 50% acetonitrile:water] to give the title compound (49 mg).

MS (ESP+): (M+H)⁺ 437.15 for C₂₀H₁₇FN₈O₃

NMR (DMSO-d₆) δ: 3.50 (t, 2H); 3.90 (dd, 1H); 4.25 (t, 1H); 4.50 (s, 3H); 4.95 (m, 1H); 6.03 (s, 1H); 6.61 (t, 1H); 7.52 (dd, 1H); 7.72 (dd, 1H); 7.78 (t, 1H); 8.20 to 8.27 (m, 2H); 8.42 (s, 1H); 8.96 (s, 1H).

### EXAMPLE 3

### (5S)-3-{3-Fluoro-4-[6-(2-methyl-2H-tetrazol-5-yl)Pyridin-3-yl]phenyl}-5-[(1,2,5-thiadiazol-3-ylamino)methyl]-1,3-oxazolidin-2-one

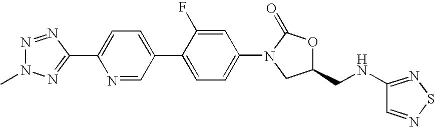 tert-Butyl [((5R)-3-{3-fluoro-4-[6-(2-methyl-2H-tetrazol-5-yl)pyridin-3-yl]phenyl}-2-oxo-1,3-oxazolidin-5-yl)methyl]1,2,5-thiadiazol-3-ylcarbamate (73.0 mg, 0.13 mmol) was dissolved in dichloromethane (10 ml) and trifluoroacetic acid (2 ml). The reaction mixture was stirred at room temperature for 2 hours. The reaction mixture was concentrated in vacuo. The residue was partitioned between dichloromethane (100 ml) and aqueous saturated sodium hydrogen carbonate solution (100 ml). The organic layer was dried over magnesium sulphate, filtered and then concentrated in vacuo onto silica gel (0.5 ml). The resulting solid was subjected to chromatography (SiO₂ 5 g bond elut column, 0 to 10% methanol/dichloromethane) to yield 57.3 mg (95%) of (5S)-3-{3-fluoro-4-[6-(2-methyl-2H-tetrazol-5-yl)pyridin-3-yl]phenyl}-5-[(1,2,5-thiadiazol-3-ylamino)methyl]-1,3-oxazolidin-2-one as a pink solid.

MS (ESP+): (M+H)⁺ 454.12 for C₁₉H₁₆FN₉O₂S

NMR (DMSO-d₆) δ: 3.93 (q, 1H), 3.63 to 3.77 (m, 2H), 4.27 (t, 1H), 4.50 (s, 3H), 4.99 (m, 1H); 7.53 (dd, 1H), 7.71 (dd, 1H); 7.75 to 7.83 (m, 1H); 8.08 (s, 1H), 8.21 to 8.28 (m, 2H), 8.96 (s, 1H).

The intermediates for this compound were prepared as follows:

### tert-Butyl [((5R)-3-{3-fluoro-4-[6-(2-methyl-2H-tetrazol-5-yl)pyridin-3-yl]phenyl}-2-oxo-1,3-oxazolidin-5-yl)methyl]1,2,5-thiadiazol-3-ylcarbamate

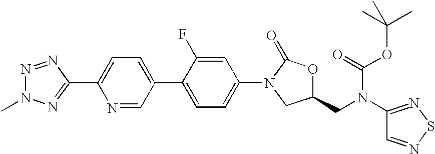 tert-Butyl {[(5R)-3-(3-fluoro-4-iodophenyl)-2-oxo-1,3-oxazolidin-5-yl]methyl}1,2,5-thiadiazol-3-ylcarbamate (304 mg, 0.584 mmol), 2-(2-methyl-2H-tetrazol-5-yl)-5-(trimethylstannyl)pyridine (cf. Example 1) (208 mg, 0.642 mmol) and copper (I) iodide (46 mg, 0.233 mmol) were dissolved in dry 1-methyl-2-pyrrolidinone (5 ml) and the reaction mixture placed under an atmosphere of argon. Tetrakis(triphenylphosphine)palladium(O) (67 mg, 0.058 mmol) was added and the reaction mixture stirred for 48 hours at 90° C. The reaction mixture was cooled to room temperature then poured into water (100 m ml). The product was extracted into ethyl acetate (100 ml). The ethyl acetate layer was separated, dried over magnesium sulphate, filtered then concentrated in vacuo. The crude product was then dissolved in dichloromethane (2 ml) and subjected to chromatography (SiO₂ 20 g bond elut column, 50 to 100% ethyl acetate/hexane) to yield 76 mg (23%) of the desired compound as a yellow oil.

MS (ESP+): (M+H)⁺ 554.13 for C₂₄H₂₄FN₉O₄S

NMR (DMSO-d₆) δ: 1.55 (s, 9H), 4.01 (q, 1H), 4.20 (dd, 1H), 4.32 (t, 1H); 4.43 (dd, 1H), 4.51 (s, 3H); 5.12 (m, 1H); 7.54 to 7.67 (m, 2H); 7.81 (t, 1H); 8.21 to 8.28 (m, 2H); 8.97 (s, 1H); 9.03 (s, 1H).

### tert-Butyl {[(5R)-3-(3-fluoro-4-iodophenyl)-2-oxo-1,3-oxazolidin-5-yl]methyl}1,2,5-thiadiazol-3-ylcarbamate

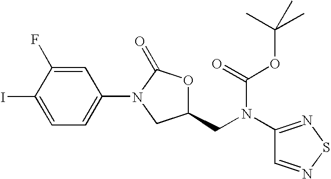 tert-Butyl 1,2,5-thiadiazol-3-ylcarbamate (860 mg, 4.23 mmol) was added as a solution in anhydrous DMF (10 ml) to a slurry of sodium hydride (60% oil dispersion, 171 mg, 4.23 mmol) in anhydrous DMF (5 ml) at room temperature. The mixture was stirred for 15 minutes then a solution of [(5R)-3-(3-fluoro-4-iodophenyl)-2-oxo-1,3-oxazolidin-5-yl]methyl methanesulfonate (1.614 g, 3.9 mmol) in anhydrous DMF (10 ml) was added dropwise over ten minutes. The reaction mixture was then stirred at 40° C. for five hours then at room temperature overnight. The reaction mixture was partitioned between ethyl acetate (200 ml) and aqueous saturated sodium hydrogen carbonate solution (200 ml). The organic layer was dried over magnesium sulphate, filtered and then concentrated in vacuo onto Isolute HM-N (15 ml) and the resulting powder was subjected to chromatography (SiO₂ 50 g bond elute columns, 0 to 25% ethyl acetate/hexane) to yield 1.202 g (59%) of tert-butyl {[(5R)-3-(3-fluoro-4-iodophenyl)-2-oxo-1,3-oxazolidin-5-yl]methyl}1,2,5-thiadiazol-3-ylcarbamate as a white solid.

NMR (DMSO-d₆) δ: 1.52 (s, 9H), 3.91 (q, 1H), 4.14 to 4.16 (m, 2H), 4.40 (dd, 1H), 5.06 (m, 1H); 7.24 (dd, 1H), 7.56 (dd, 1H); 7.86 (t, 1H); 9.01 (s, 1H).

1. A compound of the formula (I), or a pharmaceutically-acceptable salt, or an in-vivo-hydrolysable ester thereof, 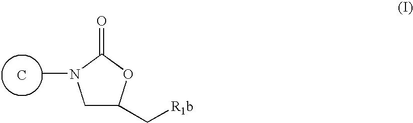
wherein C is selected from D and E, 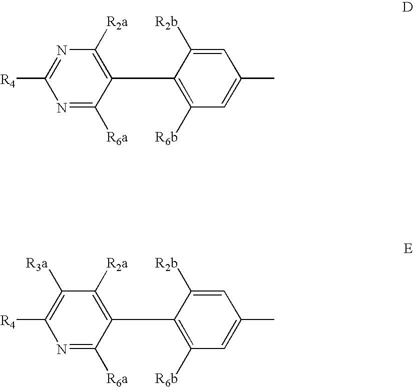
wherein in D and E the phenyl ring is attached to the oxazolidinone in (I);
R₁b is —NRz-Z wherein Rz is hydrogen, (1-6C)alkyl or —COOR₅ wherein R₅ is (1-6C) alkyl optionally substituted by one or more chlorine atoms;
Z is HET-1 wherein
HET-1 is selected from HET-LA and HET-1B wherein:
HET-1A is a C-linked 5-membered heteroaryl ring containing 2 to 4 heteroatoms independently selected from N, O and S; which ring is optionally substituted on a C atom by an oxo or thioxo group; and/or which ring is optionally substituted on any available C atom by one or two substituents selected from RT as hereinafter defined and/or on an available nitrogen atom, (provided that the ring is not thereby quaternised) by (1-4C)alkyl;
HET-1B is a C-linked 6-membered heteroaryl ring containing 2 or 3 nitrogen heteroatoms, which ring is optionally substituted on a C atom by an oxo or thioxo group; and/or which ring is optionally substituted on any available C atom by one, two or three substituents selected from RT as hereinafter defined and/or on an available nitrogen atom, (provided that the ring is not thereby quaternised) by (1-4C)alkyl;
RT is selected from a substituent from the group:
(RTa1) hydrogen, halogen, (1-4C)alkoxy, (2-4C)alkenyloxy, (2-4C)alkenyl, (2-4C)alkynyl, (3-6C)cycloalkyl, (3-6C)cycloalkenyl, (1-4C)alkylthio, amino, azido, cyano and nitro; or
(RTa2) (1-4C)alkylamino, di-(1-4C)alkylamino, and (2-4C)alkenylamino; or RT is selected from the group
(RTb1) (1-4C)alkyl group which is optionally substituted by one substituent selected from hydroxy, (1-4C)alkoxy, (1-4C)alkylthio, cyano and azido; or
(RTb2) (1-4C)alkyl group which is optionally substituted by one substituent selected from (2-4C)alkenyloxy, (3-6C)cycloalkyl, and (3-6C)cycloalkenyl; or RT is selected from the group
(RTc) a fully saturated 4-membered monocyclic ring containing 1 or 2 heteroatoms independently selected from O, N and S (optionally oxidised), and linked via a ring nitrogen or carbon atom;
and wherein at each occurrence of an RT substituent containing an alkyl, alkenyl, alkynyl, cycloalkyl or cycloalkenyl moiety in (RTa1) or (RTa2), (RTb1) or (RTb2), or (RTc) each such moiety is optionally substituted on an available carbon atom with one, two, three or more substituents independently selected from F, Cl, Br, OH and CN;
R₂a and R₆a are independently selected from H, CF₃, OMe, SMe, Me and Et;
R₂b and R₆b are independently selected from H, F, Cl, CF₃, OMe, SMe, Me and Et;
R₃a is selected from H, (1-4C)alkyl, cyano, Br, F, Cl, OH, (1-4C)alkoxy, —S(O)ₙ(1-4C)alkyl (wherein n=0, 1, or 2), amino, (1-4C)alkylcarbonylamino, nitro, —CHO, —CO(1-4C)alkyl, —CONH₂ and —CONH(1-4C)alkyl;
R₄ is selected from R₄a and R₄b wherein
R₄a is selected from azido, —NR₇R₈, OR₁₀, (1-4C)alkyl, (1-4C)alkoxy, (3-6C)cycloalkyl, —(CH₂)ₖ—R₉, AR1, AR2, (1-4C)alkanoyl, —CS(1-4C)alkyl, —C(═W)NRvRw [wherein W is O or S, Rv and Rw are independently H, or (1-4C)alkyl], —(C═O)₁—R₆, —COO(1-4C)alkyl, —C═OAR1, —C═OAR2, —COOAR1, —S(O)n(1-4C)alkyl (wherein n=1 or 2), —S(O)pAR1, —S(O)pAR2 and —C(═S)O(1-4C)alkyl; wherein any (1-4C)alkyl chain may be optionally substituted by (1-4C)alkyl, cyano, hydroxy or halo;
p=0, 1 or 2;
R₄b is selected from HET-3;
R₆ is selected from hydrogen, (1-4C)alkoxy, amino, (1-4C)alkylamino and hydroxy(1-4C)alkylamino;
k is 1 or 2;
l is 1 or 2;
R₇ and R₈ are independently selected from H and (1-4C)alkyl, or wherein R₇ and R₈ taken together with the nitrogen to which they are attached can form a 5-7 membered ring optionally with an additional heteroatom selected from N, O, S(O)n (wherein n=1 or 2) in place of 1 carbon atom of the so formed ring; wherein the ring may be optionally substituted by one or two groups independently selected from (1-4C)alkyl, (3-6C)cycloalkyl, (1-4C)alkanoyl, —COO(1-4C)alkyl, —S(O)n(1-4C)alkyl (wherein n=1 or 2), AR1, AR2, —C═OAR1, —C═OAR2, —COOAR1, —CS(1-4C)alkyl, —C(═S)O(1-4C)alkyl, —C(═W)NRvRw [wherein W is O or S, Rv and Rw are independently H, or (1-4C)alkyl], —S(O)pAR1 and —S(O)pAR2; wherein any (1-4C)alkyl, (3-6C)cycloalkyl or (1-4C)alkanoyl group may be optionally substituted (except on a carbon atom adjacent to a heteroatom) by one or two substituents selected from (1-4C)alkyl, cyano, hydroxy, halo, amino, (1-4C)alkylamino and di(1-4C)alkylamino; p=0, 1 or 2;
R₉ is independently selected from R₉a to R₉d below:
R₉a: AR1, AR2, AR2a, AR2b, AR3, AR3a, AR3b, AR4, AR4a, CY1, CY2;
R₉b: cyano, carboxy, (1-4C)alkoxycarbonyl, —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1, —CS(1-4C)alkyl and —C(═S)O(1-4C)alkyl; wherein any alkyl, alkanoyl or cycloalkyl may itself optionally be substituted by cyano, hydroxy or halo)], ethenyl, 2-(1-4C)alkylethenyl, 2-cyanoethenyl, 2-cyano-2-((1-4C)alkyl)ethenyl, 2-nitroethenyl, 2-nitro-2-((1-4C)alkyl)ethenyl, 2-((1-4C)alkylaminocarbonyl)ethenyl, 2-((1-4C)alkoxycarbonyl)ethenyl, 2-(AR1)ethenyl, 2-(AR2)ethenyl, 2-(AR2a)ethenyl;
R₉c: (1-6C)alkyl {optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, (1-4C)alkylcarbonyl, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], and amino; and/or optionally substituted by one group selected from carboxy, phosphonate [phosphono, —P(O)(OH)₂, and mono- and di-(1-4C)alkoxy derivatives thereof], phosphinate [—P(OH)₂ and mono- and di-(1-4C)alkoxy derivatives thereof], cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, (1-4C)alkylamino, di((1-4C)alkyl)amino, (1-6C)alkanoylamino-, (1-4C)alkoxycarbonylamino-, N-(1-4C)alkyl-N-(1-6C)alkanoylamino-, —C(═W)NRvRw [wherein W is O or S, Rv and Rw are as hereinbefore defined], (═NORv) wherein Rv is as hereinbefore defined, (1-4C)alkylS(O)ₚNH, (1-4C)alkylS(O)ₚ-((1-4C)alkyl)N—, fluoro(1-4C)alkylS(O)ₚNH—, fluoro(1-4C)alkylS(O)ₚ((1-4C)alkyl)N—, (1-4C)alkylS(O)_{q}—, CY1, CY2, AR1, AR2, AR3, AR1-O—, AR2-O—, AR3-O—, AR1-S(O)_{q}—, AR2-S(O)_{q}—, AR3-S(O)_{q}—, AR1-NH—, AR2-NH—, AR3-NH— (p is 1 or 2 and q is 0, 1 or 2), and also AR2a, AR2b, AR3a and AR3b versions of AR2 and AR3 containing groups}; wherein any (1-4C)alkyl present in any substituent on R₉c may itself be substituted by one or two groups independently selected from cyano, hydroxy, halo, amino, (1-4C)alkylamino and di(1-4C)alkylamino, provided that such a substituent is not on a carbon adjacent to a heteroatom atom if present;
R₉d: R₁₄C(O)O(1-6C)alkyl- wherein R₁₄ is AR1, AR2, (1-4C)alkylamino, benzyloxy-(1-4C)alkyl or (1-10C)alkyl {optionally substituted as defined for (R9c)};
R₁₀ is selected from hydrogen, R₉c (as hereinbefore defined), (3-6C)alkanoyl and (1-4C)alkylsulfonyl;
HET-3 is selected from:
a) a 5-membered heterocyclic ring contining at least one nitrogen and/or oxygen in which any carbon atom is a C═O, C═N, or C═S group, wherein said ring is of the formula HET3-A to HET3-E below: 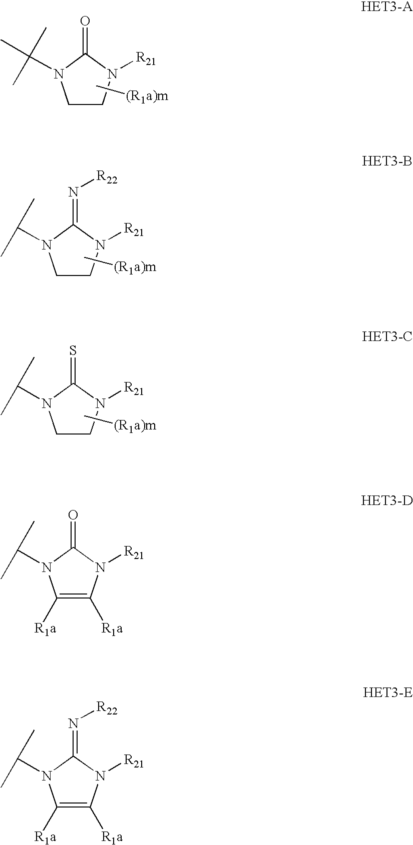
b) a carbon-linked 5- or 6-membered heteroaromatic ring containing 1, 2, 3, or 4 heteroatoms independently selected from N, O and S selected from HET3-F to HET3-Y below: 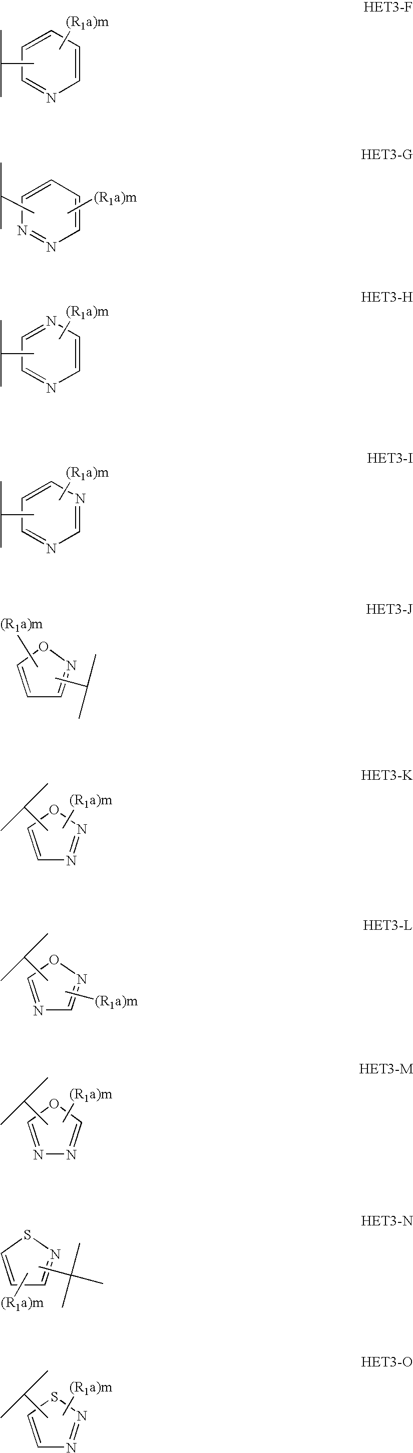 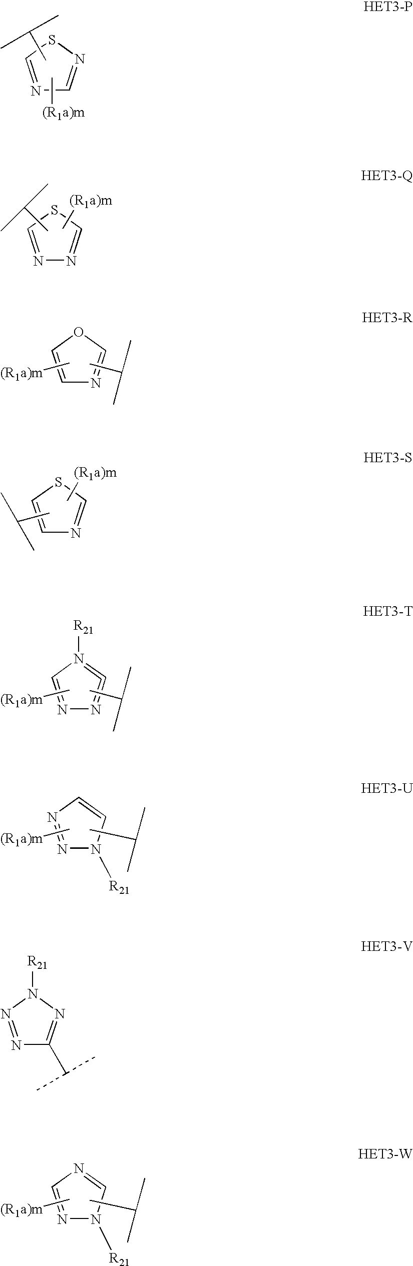 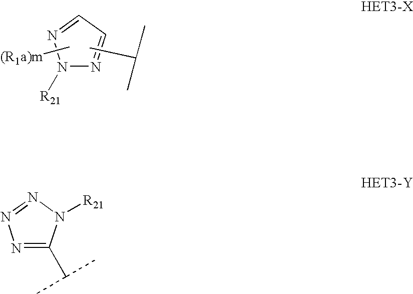
c) a nitrogen-linked 5- or 6-membered heteroaromatic ring containing 1, 2, 3, or 4 heteroatoms independently selected from N, O and S selected from HET3-Z to HET3-AH below: 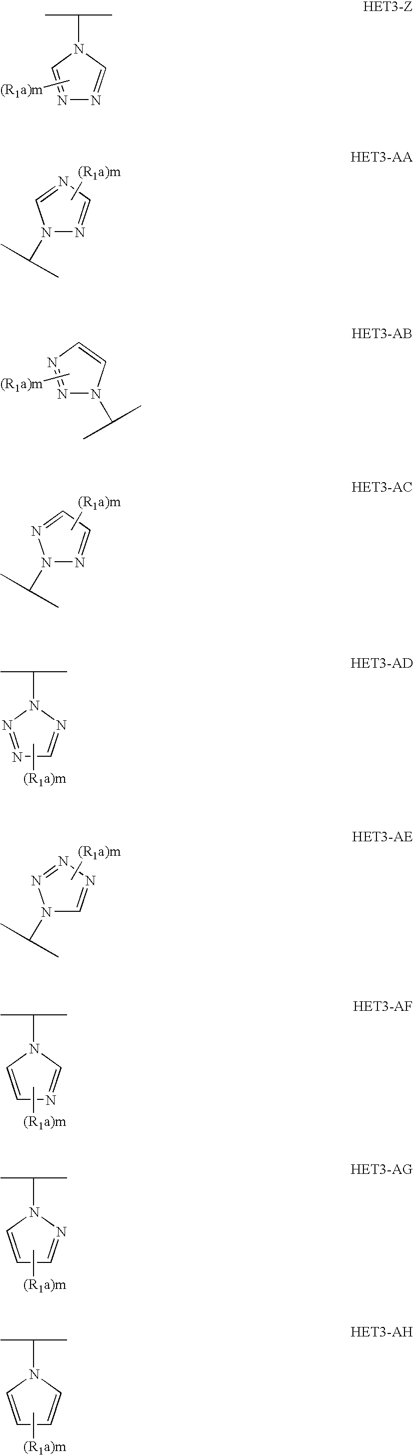
wherein in HET-3, R₁a is a substituent on carbon;
R₁a is independently selected from R₁a1 to R₁a5 below:
R₁a1: AR1, AR2, AR2a, AR2b, AR3, AR3a, AR3b, AR4, AR4a, CY1, CY2;
R₁a2: cyano, carboxy, (1-4C)alkoxycarbonyl, —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring; wherein when said ring is a piperazine ring, the ring may be optionally substituted on the additional nitrogen by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1, —CS(1-4C)alkyl) and —C(═S)O(1-4C)alkyl; wherein any (1-4C)alkyl, (1-4C)alkanoyl and (3-6C)cycloalkyl substituent may itself be substituted by cyano, hydroxy or halo, provided that, such a substituent is not on a carbon adjacent to a nitrogen atom of the piperazine ring], ethenyl, 2-(1-4C)alkylethenyl, 2-cyanoethenyl, 2-cyano-2-((1-4C)alkyl)ethenyl, 2-nitroethenyl, 2-nitro-2-((1-4C)alkyl)ethenyl, 2-((1-4C)alkylaminocarbonyl)ethenyl, 2-((1-4C)alkoxycarbonyl)ethenyl, 2-(AR1)ethenyl, 2-(AR2)ethenyl, 2-(AR2a)ethenyl;
R₁a3: (1-10C)alkyl {optionally substituted by one or more groups (including geminal disubstitution) each independently selected from hydroxy, (1-10C)alkoxy, (1-4C)alkoxy-(1-4C)alkoxy, (1-4C)alkoxy-(1-4C)alkoxy-(1-4C)alkoxy, (1-4C)alkylcarbonyl, phosphoryl [—O—P(O)(OH)₂, and mono- and di-(1-4C)alkoxy derivatives thereof], phosphiryl [—O—P(OH)₂ and mono- and di-(1-4C)alkoxy derivatives thereof], and amino; and/or optionally substituted by one group selected from carboxy, phosphonate [phosphono, —P(O)(OH)₂, and mono- and di-(1-4C)alkoxy derivatives thereof], phosphinate [—P(OH)₂ and mono- and di-(A4C)alkoxy derivatives thereof], cyano, halo, trifluoromethyl, (1-4C)alkoxycarbonyl, (1-4C)alkoxy-(1-4C)alkoxycarbonyl, (1-4C)alkoxy-(1-4C)alkoxy-(1-4C)alkoxycarbonyl, (1-4C)alkylamino, di((1-4C)alkyl)amino, (1-6C)alkanoylamino-, (1-4C)alkoxycarbonylamino-, N-(1-4C)alkyl-N-(1-6C)alkanoylamino-, —C(═W)NRvRw [wherein W is O or S, Rv and Rw are independently H, or (1-4C)alkyl and wherein Rv and Rw taken together with the amide or thioamide nitrogen to which they are attached can form a 5-7 membered ring optionally with an additional heteroatom selected from N, O, S(O)n in place of 1 carbon atom of the so formed ring;
wherein when said ring is a piperazine ring, the ring may be optionally substituted on the additional nitrogen by a group selected from (1-4C)alkyl, (3-6C)cycloalkyl, (1-4C)alkanoyl, —COO(1-4C)alkyl, —S(O)n(1-4C)alkyl (wherein n=1 or 2), —COOAR1, —CS(1-4C)alkyl and —C(═S)O(1-4C)alkyl], (═NORv) wherein Rv is as hereinbefore defined, (1-4C)alkylS(O)ₚNH—, (1-4C)alkylS(O)ₚ-((1-4C)alkyl)N—, fluoro(1-4C)alkylS(O)ₚNH—, fluoro(1-4C)alkylS(O)ₚ((1-4C)alkyl)N—, (1-4C)alkylS(O)_{q}—, CY1, CY2, AR1, AR2, AR3, AR1-O—, AR2-O—, AR3-O—, AR1-S(O)_{q}—, AR2-S(O)_{q}—, AR3-S(O)_{q}—, AR1-NH—, AR2-NH—, AR3-NH— (p is 1 or 2 and q is 0, 1 or 2), and also AR2a, AR2b, AR3a and AR3b versions of AR2 and AR3 containing groups}; wherein any (1-4C)alkyl, (1-4C)alkanoyl and (3-6C)cycloalkyl present in any substituent on R₁a3 may itself be substituted by one or two groups independently selected from cyano, hydroxy, halo, amino, (1-4C)alkylamino and di(1-4C)alkylamino, provided that such a substituent is not on a carbon adjacent to a heteroatom atom if present;
R₁a4: R₁₄C(O)O(1-6C)alkyl- wherein R₁₄ is as hereinbefore defined for R₉d;
R₁a5: F, Cl, hydroxy, mercapto, (1-4C)alkylS(O)ₚ— (p=0, 1 or 2), —NR₇R₈ (wherein R₇ and R₈ are as hereinbefore defined) or —OR₁₀ (where R₁₀ is as hereinbefore defined);
m is 0, 1 or 2;
R₂₁ is selected from hydrogen, methyl [optionally substituted with cyano, trifluoromethyl, —C═WNRvRw (where W, Rv and Rw are as hereinbefore defined for R₁a3), (1-4C)alkoxycarbonyl, (1-4C)alkoxy-(1-4C)alkoxycarbonyl, (1-4C)alkoxy-(1-4C)alkoxy-(1-4C)alkoxycarbonyl, CY1, CY2, AR1, AR2, AR2a, AR2b (not linked through nitrogen) or AR3], (2-10C)alkyl [optionally substituted other than on a carbon attached to the HET-3 ring nitrogen with one or two groups independently selected from the optional subsituents defined for R₁a3] and R₁₄C(O)O(2-6C)alkyl-, wherein R₁₄ is as defined hereinbefore and wherein R₁₄C(O)O group is attached to a carbon other than the carbon attached to the HET-3 ring nitorogen;
R₂₂ is cyano, —COR₁₂, —COOR₁₂, —CONHR₁₂, —CON(R₁₂)(R₁₃), —SO₂R₁₂ (provided that R₁₂ is not hydrogen), —SO₂NHR₁₂, —SO₂N(R₁₂)(R₁₃) or NO₂, wherein R₁₂ and R₁₃ are as defined hereinbelow;
R₁₂ and R₁₃ are independently selected from hydrogen, phenyl (optionally substituted with one or more substituents selected from halogen, (1-4C)alkyl and (1-4C)alkyl substituted with one, two, three or more halogen atoms) and (1-4C)alkyl (optionally substituted with one, two, three or more halogen atoms), or for any N(R₁₂)(R₁₃) group, R₁₂ and R₁₃ may be taken together with the nitrogen to which they are attached to form a 5-7 membered ring optionally with an additional heteroatom selected from N, O, S(O)n in place of 1 carbon atom of the so formed ring; wherein the ring may be optionally substituted by one or two groups independently selected from (1-4C)alkyl (optionally substituted on a carbon not adjacent to the nitrogen by cyano, hydroxy or halo), (3-6C)cycloalkyl, (1-4C)alkanoyl, —COO(1-4C)alkyl, —S(O)n(1-4C)alkyl (wherein n=1 or 2), AR1, AR2, —C═OAR1, —C═OAR2, —COOAR1, —CS(1-4C)alkyl, —C(═S)O(1-4C)alkyl, —C(═W)NRvRw [wherein W is O or S, Rv and Rw are independently H, or (1-4C)alkyl], —S(O)pAR1 and —S(O)pAR2; wherein any (1-4C)alkyl chain may be optionally substituted by (1-4C)alkyl, cyano, hydroxy or halo; p=0, 1 or 2;
AR1 is an optionally substituted phenyl or optionally substituted naphthyl;
AR2 is an optionally substituted 5- or 6-membered, fully unsaturated (i.e with the maximum degree of unsaturation) monocyclic heteroaryl ring containing up to four heteroatoms independently selected from O, N and S (but not containing any O—O, O—S or S—S bonds), and linked via a ring carbon atom, or a ring nitrogen atom if the ring is not thereby quaternised;
AR2a is a partially hydrogenated version of AR2 (i.e. AR2 systems retaining some, but not the full, degree of unsaturation), linked via a ring carbon atom or linked via a ring nitrogen atom if the ring is not thereby quaternised;
AR2b is a fully hydrogenated version of AR2 (i.e. AR2 systems having no unsaturation), linked via a ring carbon atom or linked via a ring nitrogen atom;
AR3 is an optionally substituted 8-, 9- or 10-membered, fully unsaturated (i.e with the maximum degree of unsaturation) bicyclic heteroaryl ring containing up to four heteroatoms independently selected from O, N and S (but not containing any O—O, O—S or S—S bonds), and linked via a ring carbon atom in either of the rings comprising the bicyclic system;
AR3a is a partially hydrogenated version of AR3 (i.e. AR3 systems retaining some, but not the full, degree of unsaturation), linked via a ring carbon atom, or linked via a ring nitrogen atom if the ring is not thereby quaternised, in either of the rings comprising the bicyclic system;
AR3b is a fully hydrogenated version of AR3 (i.e. AR3 systems having no unsaturation), linked via a ring carbon atom, or linked via a ring nitrogen atom, in either of the rings comprising the bicyclic system;
AR4 is an optionally substituted 13- or 14-membered, fully unsaturated (i.e with the maximum degree of unsaturation) tricyclic heteroaryl ring containing up to four heteroatoms independently selected from O, N and S (but not containing any O—O, O—S or S—S bonds), and linked via a ring carbon atom in any of the rings comprising the tricyclic system;
AR4a is a partially hydrogenated version of AR4 (i.e. AR4 systems retaining some, but not the full, degree of unsaturation), linked via a ring carbon atom, or linked via a ring nitrogen atom if the ring is not thereby quaternised, in any of the rings comprising the tricyclic system;
CY1 is an optionally substituted cyclobutyl, cyclopentyl or cyclohexyl ring;
CY2 is an optionally substituted cyclopentenyl or cyclohexenyl ring;
wherein; optional substituents on AR1, AR2, AR2a, AR2b, AR3, AR3a, AR3b, AR4, AR4a, CY1 and CY2 are (on an available carbon atom) up to three substituents independently selected from (1-4C)alkyl {optionally substituted by substituents selected independently from hydroxy, trifluoromethyl, (1-4C)alkyl S(O)_{q}— (q is 0, 1 or 2), (1-4C)alkoxy, (1-4C)alkoxycarbonyl, cyano, nitro, (1-4C)alkanoylamino, —CONRvRw or —NRvRw}, trifluoromethyl, hydroxy, halo, nitro, cyano, thiol, (1-4C)alkoxy, (1-4C)alkanoyloxy, dimethylaminomethyleneaminocarbonyl, di(N-(1-4C)alkyl)aminomethylimino, carboxy, (1-4C)alkoxycarbonyl, (1-4C)alkanoyl, (1-4C)alkylSO₂amino, (2-4C)alkenyl {optionally substituted by carboxy or (1-4C)alkoxycarbonyl}, (2-4C)alkynyl, (1-4C)alkanoylamino, oxo (═O), thioxo (═S), (1-4C)alkanoylamino {the (1-4C)alkanoyl group being optionally substituted by hydroxy}, (1-4C)alkyl S(O)_{q}— (q is 0, 1 or 2) {the (1-4C)alkyl group being optionally substituted by one or more groups independently selected from cyano, hydroxy and (1-4C)alkoxy}, —CONRvRw or —NRvRw [wherein Rv is hydrogen or (1-4C)alkyl; Rw is hydrogen or (1-4C)alkyl];
and further optional substituents on AR1, AR2, AR2a, AR2b, AR3, AR3a, AR3b, AR4, AR4a, CY1 and CY2 (on an available carbon atom), and also on alkyl groups (unless indicated otherwise) are up to three substituents independently selected from trifluoromethoxy, benzoylamino, benzoyl, phenyl {optionally substituted by up to three substituents independently selected from halo, (1-4C)alkoxy or cyano}, furan, pyrrole, pyrazole, imidazole, triazole, pyrimidine, pyridazine, pyridine, isoxazole, oxazole, isothiazole, thiazole, thiophene, hydroxyimino(1-4C)alkyl, (1-4C)alkoxyimino(1-4C)alkyl, halo-(1-4C)alkyl, (1-4C)alkanesulfonamido, —SO₂NRvRw [wherein Rv is hydrogen or (1-4C)alkyl; Rw is hydrogen or (1-4C)alkyl]; and
optional substituents on AR2, AR2a, AR2b, AR3, AR3a, AR3b, AR4 and AR4a are (on an available nitrogen atom, where such substitution does not result in quaternization) (1-4C)alkyl, (1-4C)alkanoyl {wherein the (1-4C)alkyl and (1-4C)alkanoyl groups are optionally substituted by (preferably one) substituents independently selected from cyano, hydroxy, nitro, trifluoromethyl, (1-4C)alkyl S(O)_{q}— (q is 0, 1 or 2), (1-4C)alkoxy, (1-4C)alkoxycarbonyl, (1-4C)alkanoylamino, —CONRvRw or —NRvRw [wherein Rv is hydrogen or (1-4C)alkyl; Rw is hydrogen or (1-4C)alkyl]}, (2-4C)alkenyl, (2-4C)alkynyl, (1-4C)alkoxycarbonyl or oxo (to form an N-oxide).

2. The compound of claim 1, wherein R1b is HET1 wherein HET1 is selected from the structures (Za) to (Zf), 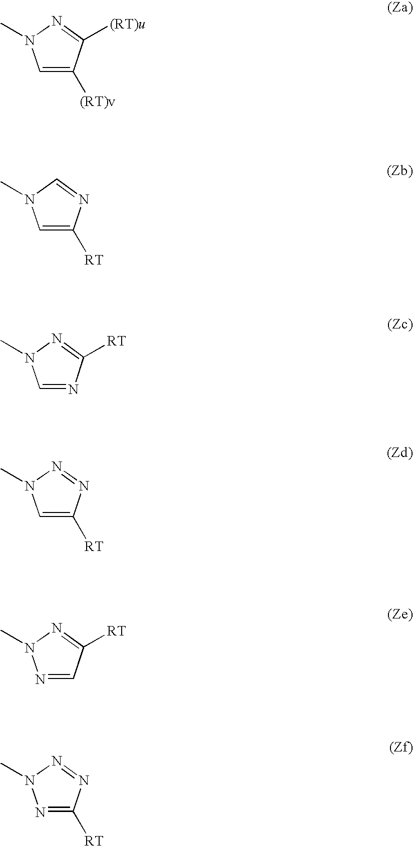
wherein u and v are independently 0 or 1 and RT is selected from:
(a) hydrogen;
(b) halogen;
(c) cyano;
(d) (1-4C)alkyl;
(e) monosubstituted (1-4C)alkyl;
(f) disubstituted (1-4C)alkyl, and
(g) trisubstituted (1-4C)alkyl.

3. The compound of claim 1, wherein R₄ is R₄b.

4. The compound of claim 1, wherein HET-3 is selected from HET3-T, HET3-V, HET3-Y and HET-3-W.

5. The compound of claim 1, wherein HET-3 is selected from HET3-V and HET3-Y.

6. The compound of claim 1, wherein R₁a is R₁a3.

7. The compound claim 1, wherein group C is group D.

8. The compound of claim 1, wherein group C is group E.

9. The compound of the formula (Ia), or a pharmaceutically-acceptable salt, or an in-vivo hydrolysable ester thereof, wherein C and R₁b have meanings as stated in claim 1.

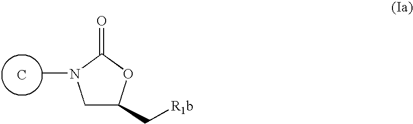

10. A pro-drug of a compound of claim 1.

11. A method for producing an antibacterial effect in a warm blooded animal which comprises administering to said animal an effective amount of a compound of claim 1.

12. (canceled)

13. (canceled)

14. A pharmaceutical composition which comprises a compound of the invention as claimed in claim 1 or a pharmaceutically-acceptable salt or an in-vivo hydrolysable ester thereof, and a pharmaceutically-acceptable diluent or carrier.

15. A process for the preparation of a compound of formula (I) as claimed in claim 1 or pharmaceutically acceptable salts or in-vivo hydrolysable esters thereof, which process comprises one of processes (a) to (f); and thereafter if necessary:
i) removing any protecting groups;
ii) forming a pro-drug (for example an in-vivo hydrolysable ester); and/or
iii) forming a pharmaceutically-acceptable salt;
wherein said processes (a) to (f) are:
a) by modifying a substituent in, or introducing a substituent into another compound of the invention;
b) by reaction of a molecule of a compound of formula (IIa) [wherein X is a leaving group useful in palladium coupling and A is either N or C—R₃a] with a molecule of a compound of formula (IIb) (wherein X′ is a leaving group useful in palladium coupling) wherein X and X′ are such that an aryl-aryl, heteroaryl-aryl, or heteroaryl-heteroaryl bond replaces the aryl-X (or heteroaryl-X) and aryl-X′ (or heteroaryl-X′) bonds; and X and X′ are chosen to be different to lead to the desired cross-coupling products of formula (I); 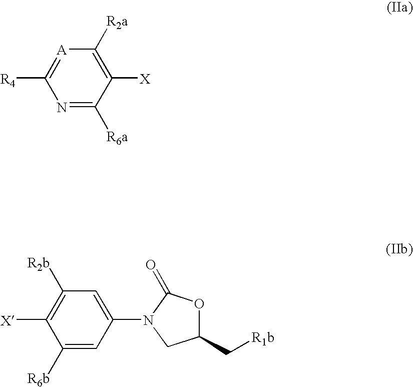
c) by reaction of a heterobiaryl derivative (III) carbamate [where A is either N or C—R₃a] with an appropriately substituted oxirane to form an oxazolidinone ring; 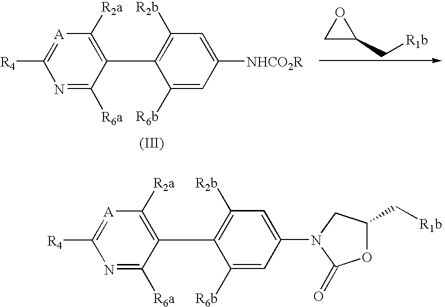
(d) by reaction of a compound of formula (VI): 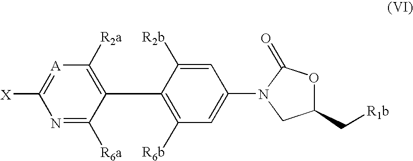
where X is a replaceable substituent with a compound of the formula (VII):
T-X′ (VII)
wherein T-X′ is HET1 or HET2 as herein above defined and X′ is a replaceable C-linked substituent; wherein the substituents X and X′ are chosen to be complementary pairs of substituents suitable as complementary substrates for coupling reactions catalysed by transition metals such as palladium(0);
(d(i)) by reaction catalysed by transition metals such as palladium(0) of a compound of formula (VIII): 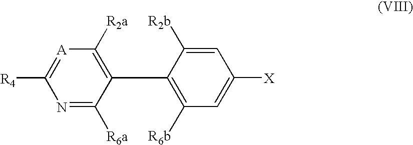
wherein X is a replaceable substituent with a compound of the formula (IX); 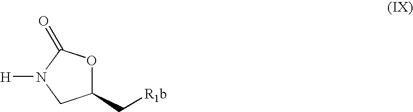
(d(ii)) by reaction of a compound of formula (X): 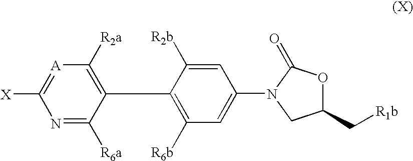
wherein X is a replaceable substituent and wherein A is either N or C—R₃a, with a compound of the formula (XI):
T-H (XI)
wherein T-H is an amine R₇R₈NH, an alcohol R₁₀OH, or an azole with an available ring-NH group to give compounds (XIIa), (XIIb), or (XIIc) wherein in this instance A is nitrogen or C—R₃a and A′ is nitrogen or carbon optionally substituted with one or more groups R₁a; 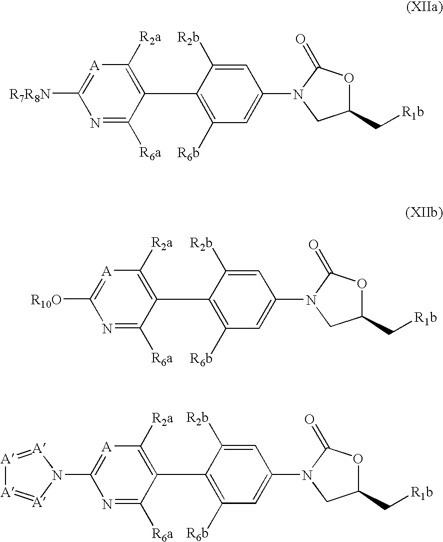
(e) by reaction of a compound of formula (XIII): 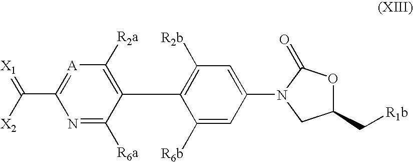
wherein X₁ and X₂ here are independently optionally substituted heteroatoms drawn in combination from O, N, and S such that C(X₁)X₂ constitutes a substituent that is a carboxylic acid derivative substituent with a compound of the formula (XIV) and X₃ and X₄ are independently optionally substituted heteroatoms drawn in combination from O, N, and S: 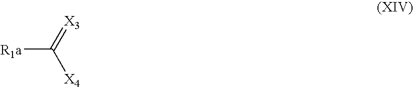
and wherein one of C(X₁)X₂ and C(X₃)X₄ constitutes an optionally substituted hydrazide, thiohydrazide, or amidrazone, hydroximidate, or hydroxamidine and the other one of C(X₁)X₂ and C(X₃)X₄ constitutes an optionally substituted acylating, thioacylating, or imidoylating agent such that C(X₁)X₂ and C(X₃)X₄ may be condensed together to form a 1,2,4-heteroatom 5-membered heterocycle containing 3 heteroatoms drawn in combination from O, N, and S, for instance thiadiazole;
(e(i)) by reaction of a compound of formula (XV): 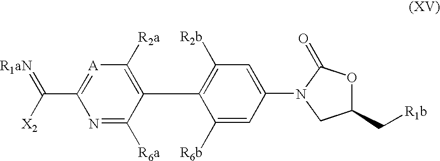
wherein X₂ is a displaceable group with a source of azide anion to give a tetrazole (XVI); 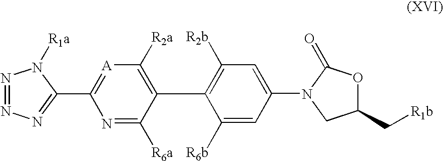
or nitriles of formula (XVII) 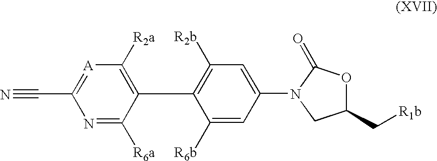
may be reacted directly with azides to give tetrazoles (XVI, R₁a=H) that are subsequently alkylated with groups R₁a≠H to give tetrazoles (XVIIIa) and (XVIIIb); 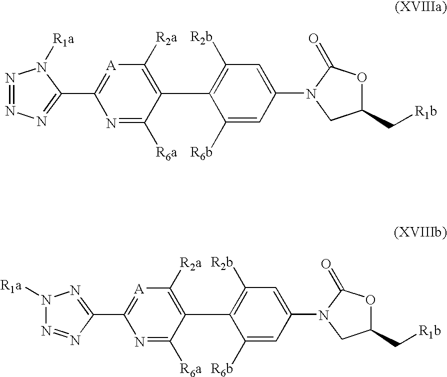
(f) by reaction of a compound of formula (XIX): 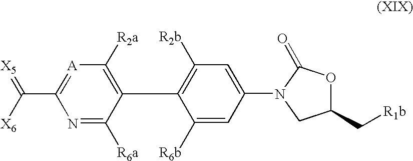
with a compound of the formula (XX): 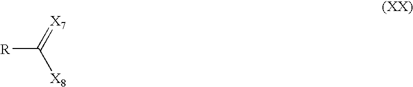
wherein one of C(X₅)X₆ and C(X₇)X₈ constitutes an optionally substituted alpha-(leaving-group-substituted)ketone, and the other one of C(X₅)X₆ and C(X₇)X₈ constitutes an optionally substituted amide, thioamide, or amidine, such that C(X₅)X₆ and C(X₇)X₈ are groups that may be condensed together to form a 1,3-heteroatom 5-membered heterocycle containing 2 heteroatoms drawn in combination from O, N, and S.
